(19) United States
(12) Patent Application Publication
(10) Pub. No.: US 2015/0366976 A1
(43) Pub. Date: Dec. 24, 2015

(54) INJECTABLE FILLER

(71) Applicants: Phi Nguyen, Houston, TX (US); Loc Phan, San Jose, CA (US); Bao Tran, Saratoga, CA (US)

(72) Inventors: Phi Nguyen, Houston, TX (US); Loc Phan, San Jose, CA (US); Bao Tran, Saratoga, CA (US)

(73) Assignee: MIBA MEDICAL INC., San Jose, CA (US)

(21) Appl. No.: 14/841,808

(22) Filed: Sep. 1, 2015

Related U.S. Application Data
(63) Continuation-in-part of application No. 13/353,316, filed on Jan. 18, 2012.
(60) Provisional application No. 62/174,525, filed on Jun. 12, 2015.
(60) Provisional application No. 61/558,669, filed on Nov. 11, 2011.

(51) Int. Cl. A61K 47/36; A61K 8/04; A61K 8/73; A61K 9/107; A61K 9/06; A61K 9/50; A61L 27/52; A61L 27/54; A61L 27/58; A61L 27/34; A61L 27/20; A61K 31/575; A61K 47/34; A61K 31/436; A61K 31/167; A61K 47/32; A61Q 19/08

(52) U.S. Cl. CPC A61K 47/36; A61Q 19/08; A61K 8/042; A61K 8/735; A61K 9/107; A61K 9/06; A61K 9/5089; A61L 27/52; A61L 27/54; A61L 27/58; A61L 27/34; A61L 27/20; A61K 31/575; A61K 47/34; A61K 31/436; A61K 31/167; A61K 47/32; A61L 2400/18; A61L 2420/02; A61L 2400/06; A61L 2300/216; A61K 2800/87; A61K 2800/91; A61K 2800/654; A61K 2800/54; A61K 2800/412

(57) ABSTRACT

A method for producing an HA gel slurry having a plurality of cross-linked units each formed by providing an inner core using a non-biological synthesis process; and cross-linking at a first cross-link strength using a hyaluronic acid (HA) or glycosaminoglycan (GAG) made from a biological synthesis process followed by additional cross-linkings at a second cross-link strength with HA or GAG, wherein the first cross-link strength is stronger than the second cross-link strength.

This application claims priority to Provisional Application Ser. No. 61/558,669 filed Nov. 11, 2011, and Utility application Ser. No. 13/301,785, filed Nov. 22, 2011, and Ser. No. 13/353,316 filed Jan. 18, 2012, the contents of which are incorporated by reference.

### BACKGROUND

The present invention relates to biocompatible viscoelastic polymeric gel slurries, methods for their preparation, formulations containing them, and medical uses thereof.

As a person age, facial rhytids (wrinkles) and folds develop in respond to the loss of facial fat and the decrease of the skin elasticity. Physicians have over the years tried various methods and materials to combat the facial volume loss of the soft tissue of the face. One of the most common methods is autologous fat transfer. Using this surgical method, a person's own fat is harvested from a different part of the body such as the abdomen, and then the fat is processed and prepared for injection into the dermal and soft tissue areas of the face that is requiring the volume restoration to alleviate the wrinkles and folds to achieve a more youthful appearance. Autologous fat transfer has good desirable results, however, this surgical technique is costly, painful, time consuming, has a long recovery time for the patient, and is associated with complications associated with any surgical procedure.

In the late 1990's, injectable fillers were introduce as an effective alternatives to the autologous fat transfer. Bovine Collagen was use as an injectable filler and was widely accepted as a less costly, less painful, quicker non surgical procedure, with faster recovery time, and has fewer associated complications. However, bovine collagen can cause an allergic respond in a small percentage of individuals and the cosmetic effects was short lived only last three to four months.

Scientists and physicians are constantly searching for the ideal dermal filler. This ideal filler should be safe and effective, biocompatible, non-immunogenic, easy to distribute and store, and should require no allergy testing. Moreover, it should be low cost, have an acceptable persistency and be easy to remove if necessary.

Hyaluronic acid (HA) dermal fillers have most of these ideal characteristics and can easily be removed whenever the practitioner considers necessary by injecting commercially available hydrolyzing specie such as hyaluronidase into the concerned area. Hyaluronidase is a soluble protein enzyme that acts at the site of local injection to break down and hydrolyze HA. Several HA fillers are currently commercially available in the US (Table 1) for mid to deep dermal implantation for the correction of moderate to severe facial wrinkles and folds, such as nasolabial folds. Hylaform® was approved in April 2004 (Monheit 2004). This HA filler is composed of HA derived from avian sources and crosslinked with divinyl sulfone (Narins and Bowman 2005). The utilization of Hylaform® dermal filler has substantially diminished since the approval of other HA fillers. Captique® dermal filler is based on non-animal HA and was approved in December 2004. Marketed by Allergan Inc., it will no longer be available after this year (2011).

A widely used dermal filler in North America is Restylane®. Restylane® was FDA-approved in December of 2003. Since 2003, with the results from the pivotal multicenter, double-blind clinical study, it has been proven that Restylane® is safe and effective in the treatment of nasolabial folds. Perlane®, a more viscous version of Restylane®, was FDA-approved in 2007. Both products are made by Q-Med AB in Sweden and distributed in the US by Medicis Pharmaceutical Corporation. They are based on “non-animal stabilized hyaluronic acid” (NASHA) and produced from cultures of Streptococcus equi via a proprietary process crosslinked with 1,4-butanediol diglycidyl ether (BDDE). The crosslinked HA is typically formulated with phosphate buffered saline in a final concentration of 20 mg/mL. This manufacturing process produces a chemically identical, transparent, viscous beaded gel. Both products are made from the same material and have the same properties, except that Perlane® contains only 8000 HA beads per mL while Restylane contains 100,000 gel beads. Restylane® and Perlane® degradation is isovolemic, meaning, it retains most of its initial filler volume throughout the degradation phase. The benefit produced by these fillers is via a volume effect and by attracting and binding water. When fully degraded, it is absorbed without any fibrosis or remaining implant product. Metabolism by-products are water and carbon dioxide. Recent histopathological research with Restylane® has shown that it also stimulates neocollagenesis (Wang et al 2007).

The new HA dermal fillers, Juvéderm™ Ultra and Juvéderm™ Ultra Plus injectable gels, are distributed by Allergan, Inc. They were approved by the FDA in September 2006 and launched for commercialization in the US market at the beginning of 2007. Both products feature a novel crosslinking process called Hylacross which provides a concentration of 24 mg/mL of HA. Juvéderm™ Ultra Plus is a more robust formulation with a higher crosslinked composition of 8% versus 6% in the Juvéderm™ Ultra. This formulation produces a softer, more viscous, non-beaded gel which is intended to enhance durability. A prospective double-blind, randomized, within-subject controlled, multi-center clinical trial comparing Juvéderm™ Ultra or Juvéderm™ Ultra Plus to bovine collagen have shown an increased persistence for the HA products (Package Insert Juvéderm Ultra L040-04 12/06; Juvéderm Ultra Plus L041-04 12/06). Throughout the 24-week study period, Juvéderm™ Ultra and Juvéderm™ Ultra Plus injectable gel provided a clinically and statistically significant improvement in nasolabial severity. Based on new clinical data demonstrating that the effects with a single treatment of either formulations may last for up to 12 months, the FDA have granted a label extension for Juvéderm™ Ultra and Juvéderm™ Ultra Plus in June, 2007 (Allergan, Inc. 2007).

Elevess™ is the latest HA approved by the FDA, in July 2007. The product, manufactured by Anika Therapeutics, MA, USA, is based on chemically modified non-animal HA proprietary technology which incorporates 0.3% lidocaine hydrochloride as a component of the treatment syringe. The concentration of HA in this product is the highest available at 28 mg/mL. Elevess™ crosslinker is p-phenylene bisethyl carbodimide (BCDI). At time of publication, this product is not commercially available.

All of these HA fillers available in the US are approved for the cosmetic improvement of the nasolabial fold; however, used off-label, injectable HA dermal fillers are useful for restoring volume to localized areas such as the cheeks, as well as reduction of the oral commissures, marionette lines, forehead lines, temple areas, tear trough, jowls, and lips.

The HA dermal fillers on the horizon are Puragen, Puragen Plus, Prevelle, Prevelle Plus, Belotero, and Teosyal family of products. Puragen and Puragen plus are based on double crosslinked (DXL™) technology with non-animal HA chains. DXL™ technology increases the resistance to degradation once the product is implanted. Puragen Plus product will incorporate lidocaine for pain management. Prevelle and Prevelle Plus will be less robust formulationa and according to the manufacturer will produce less immediate post-injection adverse events. These four products are manufactured by Mentor Corporation, CA, USA. Belotero, manufactured by Anteis SA, Geneva, Switzerland and distributed by Merz Pharmaceutical LLC, is also based on double crosslinked technology called Cohesive Polydensified Matrix (CPM) with BDDE and nonanimal HA chains. Teosyal family of products consists of 7 formulations based on monophasic, non-animal HA, crosslinked with BDDE.

Numerous roles of HA in the body have been identified. It plays an important role in the biological organism, as a mechanical support for the cells of many tissues, such as the skin, tendons, muscles and cartilage. HA is involved in key biological processes, such as the moistening of tissues, and lubrication. It is also suspected of having a role in numerous physiological functions, such as adhesion, development, cell motility, cancer, angiogenesis, and wound healing. Due to the unique physical and biological properties of HA (including viscoelasticity, biocompatibility, biodegradability), HA is employed in a wide range of current and developing applications within ophthalmology, rheumatology, drug delivery, wound healing and tissue engineering. The use of HA in some of these applications is limited by the fact that HA is soluble in water at room temperature, i.e. about 20° C., it is rapidly degraded by hyaluronidase in the body, and it is difficult to process into biomaterials. Crosslinking of HA has therefore been introduced in order to improve the physical and mechanical properties of HA and its in vivo residence time.

U.S. Pat. No. 5,143,724 discloses a method for soft tissue augmentation which comprises implanting a drug with a biocompatible viscoelastic gel slurry comprising a two phase mixture, a first phase being a particulate biocompatible gel phase, said gel phase comprising a chemically cross-linked glycosaminoglycan, or said glycosaminoglycan chemically co-cross-linked with at least one other polymer selected from the group consisting of polysaccharides and proteins, said gel phase being swollen in a physiologically acceptable aqueous medium and being uniformly distributed in the second phase, said second phase comprising a polymer solution of a water-soluble biocompatible polymer selected from the group consisting of polysaccharides, polyvinylpyrrolidone and poly ethyleneoxide in said physiologically acceptable aqueous medium, and wherein the polymer solution in the two phase mixture constitutes from 0.01 to 99.5% and the gel phase constitutes the remainder into a part of a living body where such augmentation is desired.

U.S. Pat. No. 4,582,865 (Biomatrix Inc.) describes the preparation of crosslinked gels of HA, alone or mixed with other hydrophilic polymers, using divinyl sulfone (DVS) as the crosslinking agent. The preparation of a crosslinked HA or salt thereof using a polyfunctional epoxy compound is disclosed in EP 0 161 887 B1. Other bi- or poly-functional reagents that have been employed to crosslink HA through covalent linkages include formaldehyde (U.S. Pat. No. 4,713,448, Biomatrix Inc.), polyaziridine (WO 03/089476 A1, Genzyme Corp.), L-aminoacids or L-aminoesters (WO 2004/067575, Biosphere S.P.A.). Carbodiimides have also been reported for the crosslinking of HA (U.S. Pat. No. 5,017,229, Genzyme Corp.; U.S. Pat. No. 6,013,679, Anika Research, Inc). Total or partial crosslinked esters of HA with an aliphatic alcohol, and salts of such partial esters with inorganic or organic bases, are disclosed in U.S. Pat. No. 4,957,744. Crosslinking of HA chains with 1-ethyl-3-(3-dimethylaminopropyl) carbodiimide hydrochloride (“EDAC”) and adipic acid dihydrazide in a water/acetone mixture was disclosed in U.S. 2006/0040892 (University of North Texas). WO 2006/56204 (Novozymes A/S) also discloses methods for the preparation of crosslinked gels of HA using divinyl sulfone (DVS) as the crosslinking agent.

WO 2008/100044 was published in the priority year of the present application and describes a method of preparing hyaluronic hydrogel nanoparticles by crosslinking hyaluronic acid, the method comprising mixing i) an oil phase containing a surfactant dissolved therein with ii) a water phase, containing hyaluronic acid and a water-soluble crosslinker dissolved in an aqueous basic solution where divinylsulfone is not mentioned, so as to a form a w/o emulsion, and crosslinking the hyaluronic acid in the w/o emulsion, the oil phase comprising dodecane, heptane or cetylethylhexanoate.

EP 0 830 416 (equivalent of U.S. Pat. No. 6,214,331) describes the preparation of a crosslinked water-soluble polymer particle preparation wherein the particles are less than 212 μm in diameter and wherein at least 80% of the particles are spherical, obtainable by adding an aqueous polymer solution, comprising a water-soluble polymer selected from hyaluronic acid, chondroitin sulfate, dermatan sulfate, keratan sulfate, celluloses, chitin, chitosan, agarose, carrageenans, curdlan, dextrans, emulsan, gellan, xanthans, poly(ethyleneoxide), poly(vinyl alcohol), poly(N-vinyl pyrrolidone), proteins, glycoproteins, peptidoglycans, proteoglycans, lipopolysaccharides, or combinations thereof, and an aqueous medium, to an oil base containing a water in oil emulsifying agent, agitating the mixture to form an emulsion containing polymer droplets, and crosslinking the polymer droplets in situ by a crosslinking agent resulting in the formation of crosslinked polymer particles. For the production of hyaluronic acid microspheres the crosslinking agent is added directly to an emulsion of aqueous hyaluronic acid in toluene. The crosslinking agent is first deactivated by adjusting the pH of the aqueous solution to pH 11 and then activated by lowering the pH to 7 to 8. It is preferred to use toluene, o-xylene or isooctane as oil phase. The weight ratio of aqueous phase to oil phase is about 1 to 1.

Nurettin Sahiner and Xinqiao Jai (Turk J Chem, 32 (2008), 397-409) describe the preparation of hyaluronic acid based submicron hydrogel particles using isooctane as oil phase. For preparing the emulsion 0.54 ml of aqueous hyaluronic acid solution was added to 15 ml of isooctane, resulting in a weight ratio of aqueous phase to oil phase is higher then 10 to 1.

U.S. Application 20090155362 discloses methods of producing a homogenous hydrogel comprising hyaluronic acid, or salt thereof, crosslinked with divinylsulfone (DVS), said method comprising the steps of (a) providing an alkaline solution of hyaluronic acid, or salt thereof; (b) adding DVS to the solution of step (a), whereby the hyaluronic acid, or salt thereof, is crosslinked with the DVS to form a gel; (c) treating the gel of step (b) with a buffer, wherein the gel swells and forms a hydrogel comprising hyaluronic acid, or salt thereof, crosslinked with DVS.

U.S. Application 20100311963 discloses methods of producing crosslinked hyaluronic acid microbeads, as well as the produced microbeads, said method comprising the steps of: (a) mixing an aqueous alkaline solution comprising hyaluronic acid, or a salt thereof, with a solution comprising a crosslinking agent; (b) forming microdroplets having a desired size from the mixed solution of step (a) in an organic or oil phase to form a water in organic or water in oil (W/O) emulsion; (c) continuously stirring the W/O emulsion, whereby the reaction of hyaluronic acid with divinylsulfone takes place to provide crosslinked hyaluronic acid microbeads; and (d) purifying the crosslinked hyaluronic acid microbeads.

### SUMMARY

Systems and method are disclosed for producing an HA gel slurry having a plurality of cross-linked units each formed by providing an inner core using a non-biological synthesis process; and cross-linking at a first cross-link strength using a hyaluronic acid (HA) or glycosaminoglycan (GAG) made from a biological synthesis process followed by additional cross-linkings at a second cross-link strength with HA or GAG, wherein the first cross-link strength is stronger than the second cross-link strength.

In implementations, the inner core can be bio-compatible composition such as polymers: silicones, poly (ethylene), poly (vinyl chloride), polyurethanes, polylactides. The inner core can also be natural polymers: collagen, gelatin, elastin, silk, polysaccharide. The inner core can also be cellulose, polysaccharide, hydroxypropyl cellulose, among others. The inner core can be genetic or metabolic engineering for HA synthesis. The inner core can also be artificial (in vitro) synthesis of HA by enzymes. One embodiment for making the inner core of synthetic hyaluronic acid employs two monosaccharide glycosyl donors to create the repeating polymer. The reverse disaccharide pathway provides a hyaluronic acid-like glucose-β-(1→4)-glucosamine disaccharide, for example.

In another aspect, a method for forming a biocompatible cross-linked polymer system where the cross densities are inversely related to the interface surface of the polymer system and the its internal core. That is, from the internal core of the polymer system to the interface surfaces, the cross-linking levels decrease to nearing non-cross-linked. This biocompatible cross-linked polymer system includes cross-linking a heteropolysaccharide to form a first cross-linked (cross-linker types might be varied) material;
cross-linking the first cross-linked material using the same pre-polymer mix as that of the first or a new pre-polymer mix could be prepared containing the
cross-linker to form a second cross-linked material with an interpenetrating polymer network (IPN) between the first and second cross-linked materials; several layers might be created using the same processing technique; and
performing one or more additional cross-linkings on the second cross-linked material to form a multiply cross-linked material adding connections by intertwining to form the IPN for resisting biodegradation in a human body than the single progressively lower cross-linked material and one or more single cross-linked extensions radiating out from the IPN, wherein the combination of the IPN and the extension provide one or more of: biodegradation resistance, native tissue bio-compatibility soft touch feeling, ease of insertion into the human body.

In another aspect, methods for cosmetic augmentation inclujdes forming a biocompatible cross-linked polymer having a multi-phase mixture with a predetermined controlled release of a pharmaceutical substance to modulate soft tissue response to the polymer, the polymer having at least one phase cross-linked, glycosaminoglycan in a physiological buffer solution; and augmenting soft tissue with the biocompatible cross-linked polymer.

Other aspect includes a method of controlling adhesion formation between tissues of a living body resulting from non-surgical intervention includes forming a biocompatible cross-linked polymer having a multi-phase mixture with a strategically controlled release of a pharmaceutical substance to modulate soft tissue response to the polymer, the polymer having at least one phase cross-linked, glycosaminoglycan in a physiological buffer solution; and augmenting soft tissue with the biocompatible cross-linked polymer.

Yet another aspect includes a method of controlling cell movement and attachment to surfaces in a living body by forming a biocompatible cross-linked polymer having a multi-phase mixture with a strategic controlled release of a pharmaceutical substance to modulate soft tissue response to the polymer, the polymer having at least one phase cross-linked, glycosaminoglycan in a physiological buffer solution; and augmenting soft tissue with the biocompatible cross-linked polymer.

A further aspect includes a method for controlled drug delivery includes forming a biocompatible cross-linked polymer having a multi-phase mixture with a strategic controlled release of a pharmaceutical substance to modulate soft tissue response to the polymer, the polymer having at least one phase cross-linked, glycosaminoglycan in a physiological buffer solution; and augmenting soft tissue with the biocompatible cross-linked polymer.

Yet another aspect includes a method of viscosupplementation for medical purposes includes forming a biocompatible cross-linked polymer having a multi-phase mixture with a strategic controlled release of a pharmaceutical substance to modulate soft tissue response to the polymer, the polymer having at least one phase cross-linked, glycosaminoglycan in a physiological buffer solution; and augmenting soft tissue with the biocompatible cross-linked polymer.

Other aspect includes methods are disclosed to control the rheological and diffusion characteristics of the instant biocompatible gel slurries.

Additional aspect includes methods are disclosed for optimizing biodegradation profiles and control migration of the implant material through the manipulation of various types molecular weight

Further aspect includes methods are disclosed for an implant that feels natural to the touch.

Implementations of the above aspects may include one or more of the following. The system is biocompatible and performs controlled drug releases at strategic timing to coinside with key physiological events. For example, a fast drug release profile and no delay would be well suited for the controlled release of an anesthetic such as lidocain to relieve acute pain experienced by the patient associated with the surgical procedure. The system is also capable of a medium release profile and a medium delay of a corticosteroid or steroid such as dexamethasone or triamcinolone to co-inside with a physiological inflammatory foreign body reaction. The system can also be customized to have a medium to slow release profile and a longer delay before starting the release of an antiproliferative drug such as paclitaxel, serolimas or 5-flourouracil to stop uncontrolled healing and excessive remodeling causing unsightly scar formation. The system controls the scar formation process around a foreign body such as in capsular formation. The system optimizes biodegradation profiles and controls migration of the implant material. The system can be formulated around various types of molecular weights such as Mₙ, M_{w} and M_{z}, their dispersity (PDI) to optimize the biodegradation profiles to be from hypervolumic to isovolumic to hypovolumic. A natural feel is achieved through viscoelastic harmony of properties between the existing tissue and the implant. This can be done by manipulating the viscous component of the implant through flow properties by way of the particle size and particle size distribution ratios. The elastic component is intrinsic within the material tertiary structure (molecular weight and steric hindrance) and cross linking densities.

### DESCRIPTION

First, the preparation of the hyaluronic acid is discussed, followed by the addition of additional chemicals to provide cost-effective and improved hyaluronic for dermal or subdermal use is discussed.

The term “hyaluronic acid” is used in literature to mean acidic polysaccharides with different molecular weights constituted by residues of D-glucuronic and N-acetyl-D-glucosamine acids, which occur naturally in cell surfaces, in the basic extracellular substances of the connective tissue of vertebrates, in the synovial fluid of the joints, in the endobulbar fluid of the eye, in human umbilical cord tissue and in cocks' combs.

The term “hyaluronic acid” is in fact usually used as meaning a whole series of polysaccharides with alternating residues of D-glucuronic and N-acetyl-D-glucosamine acids with varying molecular weights or even the degraded fractions of the same, and it would therefore seem more correct to use the plural term of “hyaluronic acids”. The singular term will, however, be used all the same in this description; in addition, the abbreviation “HA” will frequently be used in place of this collective term.

“Hyaluronic acid” is defined herein as an unsulphated glycosaminoglycan composed of repeating disaccharide units of N-acetylglucosamine (GIcNAc) and glucuronic acid (GlcUA) linked together by alternating beta-1,4 and beta-1,3 glycosidic bonds. Hyaluronic acid is also known as hyaluronan, hyaluronate, or HA. The terms hyaluronan and hyaluronic acid are used interchangeably herein.

Rooster combs are a significant commercial source for hyaluronan. Microorganisms are an alternative source. U.S. Pat. No. 4,801,539 discloses a fermentation method for preparing hyaluronic acid involving a strain of Streptococcus zooepidemicus with reported yields of about 3.6 g of hyaluronic acid per liter. European Patent No. EP0694616 discloses fermentation processes using an improved strain of Streptococcus zooepidemicus with reported yields of about 3.5 g of hyaluronic acid per liter. As disclosed in WO 03/054163 (Novozymes), which is incorporated herein in its entirety, hyaluronic acid or salts thereof may be recombinantly produced, e.g., in a Gram-positive Bacillus host.

Hyaluronan synthases have been described from vertebrates, bacterial pathogens, and algal viruses (DeAngelis, P. L., 1999, Cell. Mol. Life Sci. 56: 670-682). WO 99/23227 discloses a Group I hyaluronate synthase from Streptococcus equisimilis. WO 99/51265 and WO 00/27437 describe a Group II hyaluronate synthase from Pasteurella multocida. Ferretti et al. discloses the hyaluronan synthase operon of Streptococcus pyogenes, which is composed of three genes, hasA, hasB, and hasC, that encode hyaluronate synthase, UDP glucose dehydrogenase, and UDP-glucose pyrophosphorylase, respectively (Proc. Natl. Acad. Sci. USA. 98, 4658-4663, 2001). WO 99/51265 describes a nucleic acid segment having a coding region for a Streptococcus equisimilis hyaluronan synthase.

Since the hyaluronan of a recombinant Bacillus cell is expressed directly to the culture medium, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. The hyaluronan may be dried or concentrated from the filtrate solution by using evaporative techniques known to the art, such as lyophilization or spraydrying.

The term “microbead” is used herein interchangeably with microdrop, microdroplet, microparticle, microsphere, nanobead, nanodrop, nanodroplet, nanoparticle, nanosphere etc. A typical microbead is approximately spherical and has an number average cross-section or diameter in the range of between 1 nanometer to 1 millimeter. Though, usually the microbeads of the one embodiment will be made with a desired size in a much more narrow range, i.e., they will be fairly uniform. The microbeads preferably have a diameter in the range of about 100-1,000 nanometer; or in the range of 1,000 nanometer to 1,000 micrometer. The size-distribution of the microbeads will be low and the polydispersibility narrow.

### Host Cells

A preferred embodiment relates to the method of the first aspect, wherein the hyaluronic acid or salt thereof is recombinantly produced, preferably by a Gram-positive bacterium or host cell, more preferably by a bacterium of the genus Bacillus.

The host cell may be any Bacillus cell suitable for recombinant production of hyaluronic acid. The Bacillus host cell may be a wild-type Bacillus cell or a mutant thereof. Bacillus cells useful in the practice of the one embodiment include, but are not limited to, Bacillus agaraderhens, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. Mutant Bacillus subtilis cells particularly adapted for recombinant expression are described in WO 98/22598. Non-encapsulating Bacillus cells are particularly useful in the one embodiment.

In one embodiment, the Bacillus host cell is a Bacillus amyloliquefaciens, Bacillus clausii, Bacillus lentus, Bacillus licheniformis, Bacillus stearothermophilus or Bacillus subtilis cell. In a more preferred embodiment, the Bacillus cell is a Bacillus amyloliquefaciens cell. In another more preferred embodiment, the Bacillus cell is a Bacillus clausii cell. In another more preferred embodiment, the Bacillus cell is a Bacillus lentus cell. In another more preferred embodiment, the Bacillus cell is a Bacillus licheniformis cell. In another more preferred embodiment, the Bacillus cell is a Bacillus subtilis cell. In a most preferred embodiment, the Bacillus host cell is Bacillus subtilis A164Δ5 (see U.S. Pat. No. 5,891,701) or Bacillus subtilis 168Δ4.

### Molecular Weight

The content of hyaluronic acid may be determined according to the modified carbazole method (Bitter and Muir, 1962, Anal Biochem. 4: 330-334). Moreover, the number average molecular weight of the hyaluronic acid may be determined using standard methods in the art, such as those described by Ueno et al., 1988, Chem. Pharm. Bull. 36, 4971-4975; Wyatt, 1993, Anal. Chim. Acta 272: 1-40; and Wyatt Technologies, 1999, “Light Scattering University DAWN Course Manual” and “DAWN EOS Manual” Wyatt Technology Corporation, Santa Barbara, Calif.

In one embodiment, the hyaluronic acid, or salt thereof, of the one embodiment has a molecular weight of about 10,000 to about 10,000,000 Da. In a more preferred embodiment it has a molecular weight of about 25,000 to about 5,000,000 Da. In a most preferred embodiment, the hyaluronic acid has a molecular weight of about 50,000 to about 3,000,000 Da.

In another embodiment, the hyaluronic acid or salt thereof has a molecular weight in the range of between 300,000 and 3,000,000; preferably in the range of between 400,000 and 2,500,000; more preferably in the range of between 500,000 and 2,000,000; and most preferably in the range of between 600,000 and 1,800,000.

In yet another embodiment, the hyaluronic acid or salt thereof has a low number average molecular weight in the range of between 10,000 and 800,000 Da; preferably in the range of between 20,000 and 600,000 Da; more preferably in the range of between 30,000 and 500,000 Da; even more preferably in the range of between 40,000 and 400,000 Da; and most preferably in the range of between 50,000 and 300,000 Da.

### Salts and Crosslinked HA

One embodiment relates to a method of the first aspect, which comprises an inorganic salt of hyaluronic acid, preferably sodium hyaluronate, potassium hyaluronate, ammonium hyaluronate, calcium hyaluronate, magnesium hyaluronate, zinc hyaluronate, or cobalt hyaluronate.

### Other Ingredients

In another embodiment, the product produced by the method of one embodiment may also comprise other ingredients, preferably one or more active ingredient, preferably one or more pharmacologically active substance, and also preferably a water-soluble excipient, such as lactose or a non-biologically derived sugar.

Non-limiting examples of an active ingredient or the one or more pharmacologically active substance(s) which may be used in the one embodiment include vitamin(s), anti-inflammatory drugs, antibiotics, bacteriostatics, general anaesthetic drugs, such as, lidocaine, morphine etc. as well as protein and/or peptide drugs, such as, human growth hormone, bovine growth hormone, porcine growth hormone, growth hormone releasing hormone/peptide, granulocyte-colony stimulating factor, granulocyte macrophage-colony stimulating factor, macrophage-colony stimulating factor, erythropoietin, bone morphogenic protein, interferon or derivative thereof, insulin or derivative thereof, atriopeptin-Ill, monoclonal antibody, tumor necrosis factor, macrophage activating factor, interleukin, tumor degenerating factor, insulin-like growth factor, epidermal growth factor, tissue plasminogen activator, factor IIV, factor IIIV, and urokinase.

A water-soluble excipient may be included for the purpose of stabilizing the active ingredient(s), such excipient may include a protein, e.g., albumin or gelatin; an amino acid, such as glycine, alanine, glutamic acid, arginine, lysine and a salt thereof; carbohydrate such as glucose, lactose, xylose, galactose, fructose, maltose, saccharose, dextran, mannitol, sorbitol, trehalose and chondroitin sulphate; an inorganic salt such as phosphate; a surfactant such as TWEEN® (ICI), poly ethylene glycol, and a mixture thereof. The excipient or stabilizer may be used in an amount ranging from 0.001 to 99% by weight of the product.

Several aspects of one embodiment relate to various compositions and pharmaceuticals comprising, among other constituents, an effective amount of the crosslinked HA product, and an active ingredient, preferably the active ingredient is a pharmacologically active agent; a pharmaceutically acceptable carrier, excipient or diluent, preferably a water-soluble excipient, and most preferably lactose.

In addition, aspects of one embodiment relate to articles comprising a product as defined in the first aspect or a composition as defined in the aspects and embodiments above, e.g., a sanitary article, a medical or surgical article. In a final aspect one embodiment relates to a medicament capsule or microcapsule comprising a product as defined in the first aspect or a composition as defined in other aspects and embodiments of one embodiment.

One method of producing crosslinked hyaluronic acid microbeads include:

(a) mixing an aqueous alkaline solution comprising hyaluronic acid, or a salt thereof, with a solution comprising a crosslinking agent;

(b) forming microdroplets having a desired size from the mixed solution of step (a) in an organic or oil phase to form a water in organic or water in oil (W/O) emulsion;

(c) continuously stirring the W/O emulsion, whereby the reaction of hyaluronic acid with divinylsulfone takes place to provide crosslinked hyaluronic acid microbeads; and

(d) purifying the crosslinked hyaluronic acid microbeads.

It has previously been described how to produce hyaluronic acid recombinantly in a Bacillus host cell, see WO 2003/054163, Novozymes NS, which is incorporated herein in its entirety. The hyaluronic acid, or salt thereof, can also be recombinantly produced in a Bacillus host cell. Various molecular weight fractions of hyaluronic acid have been described as advantageous for specific purposes.

One embodiment relates to a method of the first aspect, wherein the hyaluronic acid, or salt thereof, has an number average molecular weight of between 100 and 3,000 kDa, preferably between 500 and 2,000 kDa, and most preferably between 700 and 1,800 kDa. The initical concentration of hyaluronic acid, or a salt thereof, in the method of one embodiment, influences the properties of the resulting crosslinked microbeads. Therefore, one embodiment relates to a method of the first aspect, wherein the alkaline solution comprises dissolved hyaluronic acid, or salt thereof, in a concentration of between 0.1%-40% (w/v).

The pH value during the crosslinking reaction also influences the outcome, so in a preferred embodiment one embodiment relates to a method of the first aspect, wherein the alkaline solution comprises dissolved sodium hydroxide in a concentration of between 0.001-2.0 M. The concentration of the crosslinking agent has a profound impact on the resulting microbeads.

Consequently, one embodiment relates to a method of the first aspect, wherein the crosslinking agent is divinylsulfone (DVS); preferably DVS is comprised in the mixed solution of step (a) in a weight ratio of between 1:1 and 100:1 of HA/DVS (dry weight), preferably between 2:1 and 50:1 of HA/DVS (dry weight).

Other crosslinking agents are also envisioned as being suitable for the methods of the one embodiment, such as, crosslinking agents based on bisepoxide crosslinking technology: GDE=glycerol diglycidyl ether and BDE: 1,4-butanediol diglycidyl ether.

Crosslinking agents suitable for the methods of the one embodiment are for example poly functional (>=2) OH-reactive compounds. Examples for suitable crosslinking agents are divinylsulfone (DVS) or crosslinking agents based on bisepoxide crosslinking technology, for example GDE=glycerol diglycidyl ether or BDE: 1,4-butanediol diglycidyl ether. The crosslinking agent is preferably selected from divinylsulfone, glycerol diglycidyl ether or 1,4-butanediol diglycidyl ether. The most preferred crosslinking agent of one embodiment is divinylsulfone which is preferably used in the weight ratio mentioned above.

An initial period of stirring during and/or immediately after mixing the solution comprising the crosslinking agent and the HA-solution was desirable to achieve satisfactory gelling. Accordingly, one embodiment relates to a method of the first aspect, wherein the reaction of hyaluronic acid with divinylsulfone takes place at a temperature in the range of 5° C.-100° C., preferably in the range of 15° C.-50° C., more preferably in the range of 20° C.-30° C.

In another preferred embodiment, the stirring in step (c) is continued for a period of between 1-180 minutes.

A heating step can be beneficial after mixing the solutions. Accordingly, the mixed solution is heated to a temperature in the range of 20° C.-100° C., preferably in the range of 25° C.-80° C., more preferably in the range of 30° C.-60° C., and most preferably in the range of 35° C.-55° C., and the temperature is maintained in this range for a period of at least 5 minutes, preferably at least 10 minutes, 20 minutes, 30, 40, 50, 60, 70, 80, 90, 100, 110, 120, 130, 140, 150, 160, 170, or most preferably at least 180 minutes after mixing the solutions; preferably without stirring.

It is advantageous to leave the reaction mixture at room temperature for a brief period after the crosslinking reaction has taken place, but still with continuous stirring.

In one embodiment, the reaction mixture is maintained after the reaction has taken place for a period of at least 5 minutes, preferably at least 10 minutes, 20 minutes, 30, 40, 50, 60, 70, 80, 90, 100, 110, 120, 130, 140, 150, 160, 170, or most preferably at least 180 minutes, at a temperature in the range of 0° C.-40° C., preferably in the range of 10° C.-30° C. It might by advantageous when the microdroplets of step (b) have a number average diameter in the range of from about 1 nanometre to 1 millimetre. The maximum of the particle size distribution of the microdroplets of step (b) is preferably in the range of from 0.1 to 100 pm, more preferably from 0.5 to 10 μm and most preferably from 1 to 2 μm. The size of the droplets can be adjusted by the choice of emulsifier used and the intensity of stirring. The combination of emulsifier used and intensity of stirring necessary to obtain droplets with the desired size can be determined by simple test series. The microdroplets can have a number average diameter in the range of about 1 nanometer to 1 millimeter. It is also preferred that the crosslinked microbead of the second aspect has a number average diameter in the range of about 1 nanometer to 1 millimeter. It might be advantageous to obtain a dispersion in step (c) that comprises almost none unreacted crosslinking agent. Preferably the dispersion more preferably the microbeads comprise less than 10 ppm by weight (wppm), more preferably less than 5 wppm. The concentration of free crosslinking agent in the dispersion especially needs to be low if the dispersion is directly used in pharmaceutical or biomedical application/device compositions because the unreacted crosslinking agent might be a toxicological threat. It is therefore preferred to last the reaction of step (c) till a dispersion is obtained comprising the unreacted crosslinking agent in the concentration mentioned above.

Compounds from at least one of the following groups can be employed as nonionic emulsifiers or surfactants: addition products of from 2 to 100 mol of ethylene oxide and/or 0 to 5 mol of propylene oxide on linear fatty alcohols having 8 to 22 C atoms, on fatty acids having 12 to 22 C atoms and on alkylphenols having 8 to 15 C atoms in the alkyl group, C12/18-fatty acid mono- and diesters of addition products of from 1 to 100 mol of ethylene oxide on glycerol, glycerol mono- and diesters and sorbitan mono- and diesters of saturated and unsaturated fatty acids having 6 to 22 carbon atoms and ethylene oxide addition products thereof, alkyl mono- and oligoglycosides having 8 to 22 carbon atoms in the alkyl radical and ethylene oxide addition products thereof, addition products of from 2 to 200 mol of ethylene oxide on castor oil and/or hydrogenated castor oil, partial esters based on linear, branched, unsaturated or saturated C6-C22-fatty acids, ricinoleic acid and 12-hydroxystearic acid and glycerol, polyglycerol, pentaerythritol, dipentaerythritol, sugar alcohols (e.g. sorbitol), alkyl glucosides (e.g. methyl glucoside, butyl glucoside, lauryl glucoside) and polyglucosides (e.g. cellulose), mono-, di- and trialkyl phosphates and mono-, di- and/or tri-PEG-alkyl phosphates and salts thereof, polysiloxane/polyether copolymers (Dimethicone Copolyols), such as e.g. PEG/PPG-20/6 Dimethicone, PEG/PPG-20/20 Dimethicone, Bis-PEG/PPG-20/20 Dimethicone, PEG-12 or PEG-14 Dimethicone, PEG/PPG-14/4 or 4/12 or 20/20 or 18/18 or 17/18 or 15/15, polysiloxane/polyalkyl polyether copolymers and corresponding derivatives, such as e.g. Lauryl or Cetyl Dimethicone Copolyols, in particular Cetyl PEG/PPG-10/1 Dimethicone (ABIL® EM 90 (Evonik Degussa)), mixed esters of pentaerythritol, fatty acids, citric acid and fatty alcohol according to DE 11 65 574 and/or mixed esters of fatty acids having 6 to 22 carbon atoms, methylglucose and polyols, such as e.g. glycerol or polyglycerol, citric acid esters, such as e.g. Glyceryl Stearate Citrate, Glyceryl Oleate Citrate and Dilauryl Citrate.

Preferred emulsifiers used in the one embodiment are selected from those having a HLB-value of from 3 to 9, preferably 4 to 6 and more preferably about 5. Preferred emulsifiers are selected from polyglyceryl-4-diisostearat/polyhydroxysterat/sebacat (ISOLAN® GPS), PEG/PPG-10/1 dimethicone, (ABIL® EM 90), Polyglyceryl-4 Isostearate (ISOLAN® GI 34), Polyglyceryl-3 Oleate (ISOLAN® GO 33), Methylglucose Isostearate (ISOLAN® IS), Diisostearoyl Polyglyceryl-3 Dimer Dilinoleate (ISOLAN® PDI), Glyceryl Oleate (TEGIN® O V), Sorbitan Laurate (TEGO® SML), Sorbitan Oleate (TEGO® SMO V) and Sorbitan Stearate (TEGO® SMS). These preferred emulsifiers are available from Evonik Goldschmidt GmbH.

Anionic emulsifiers or surfactants can contain groups which confer solubility in water, such as e.g. a carboxylate, sulphate, sulphonate or phosphate group and a lipophilic radical. Anionic surfactants which are tolerated by skin are known in large numbers to the person skilled in the art and are commercially obtainable. In this context these can be alkyl sulphates or alkyl phosphates in the form of their alkali metal, ammonium or alkanolammonium salts, alkyl ether-sulphates, alkyl ether-carboxylates, acyl sarcosinates and sulphosuccinates and acyl glutamates in the form of their alkali metal or ammonium salts.

Cationic emulsifiers and surfactants can also be added. Quaternary ammonium compounds, in particular those provided with at least one linear and/or branched, saturated or unsaturated alkyl chain having 8 to 22 C atoms, can be employed in particular as such, thus, for example, alkyltrimethylammonium halides, such as e.g. cetyltrimethylammonium chloride or bromide or behenyltrimethylammonium chloride, but also dialkyldimethylammonium halides, such as e.g. distearyldimethylammonium chloride.

Monoalkylamidoquats, such as e.g. palmitamidopropyltrimethylammonium chloride, or corresponding dialkylamidoquats can furthermore be employed. Readily biodegradable quaternary ester compounds, which can be quaternized fatty acid esters based on mono-, di- or triethanolamine, can furthermore be employed. Alkylguanidinium salts can furthermore be admixed as cationic emulsifiers.

Typical examples of mild surfactants, i.e. surfactants which are particularly tolerated by skin, are fatty alcohol polyglycol ether-sulphates, monoglyceride sulphates, mono- and/or dialkyl sulphosuccinates, fatty acid isethionates, fatty acid sarcosinates, fatty acid taurides, fatty acid glutamates, ether-carboxylic acids, alkyl oligoglucosides, fatty acid glucamides, alkylamidobetaines and/or protein-fatty acid condensates, the latter for example based on wheat proteins.

It is furthermore possible to employ amphoteric surfactants, such as e.g. betaines, amphoacetates or amphopropionates, thus e.g. substances such as the N-alkyl-N, N-dimethylammonium glycinates, for example coco-alkyldimethylammonium glycinate, N-acylaminopropyl-N,N-dimethylammonium glycinates, for example coco-acylamimopropyldimethylammonium glycinate, and 2-alkyl-3-carboxymethyl-3-hydroxyethylimidazolines having in each case 8 to 18 C atoms in the alkyl or acyl group, and coco-acylaminoethylhydroxyethylcarboxymethyl glycinate.

Of the ampholytic surfactants, those surface-active compounds which contain, apart from a C8/18-alkyl or -acyl group, at least one free amino group and at least one —COOH or —SO3H group in the molecule and are capable of formation of inner salts can be employed. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 C atoms in the alkyl group. Further examples of ampholytic surfactants are N-coco-alkylaminopropionate, coco-acylaminoethylaminopropionate and 012/18-acrylsarcosine.

Preferred emulsifiers or surfactants used for formulating the composition are identical to those used in the production of the microbeads.

Many types of buffers or acids, as are well known to the skilled person, have been envisioned as suitable for the swelling and neutralizing of the crosslinked microbeads of one embodiment. In a preferred embodiment the buffer comprises a buffer with a pH value in the range of 2.0-8.0, preferably in the range of 5.0-7.5.

Optimally, a suitable buffer is chosen with a pH value, which results in that the crosslinked microbeads have a pH value as close to neutral as possible. In one embodiment, the buffer comprises a buffer with a pH value, which results in that the crosslinked microbeads have a pH value between 5.0 and 7.5. The buffer can be a phosphate buffer and/or a saline buffer. The crosslinked microbeads can be washed at least once with water, water and an acid, water and a phosphate buffer, water and a saline buffer, or water and a phosphate buffer and a saline buffer, with a pH value in the range of 2.0-8.0, preferably in the range of 5.0-7.5. The purifying step may comprise any separation technique known in the art, e.g. filtration, decantation, centrifugation and so on. It might be advantageous to combine one or more purifying steps with one or more neutralizing steps.

The purifying step can include dialyzing the crosslinked microbeads against de-ionized water using a dialysis membrane that allows free diffusion of molecules having a size less than 13,000 Daltons. Standard emollients used in cosmetic or personal care formulations as oil phase can be added. Such standard emollients are not hydrocarbons or aromatic hydrocarbons, especially not toluene, o-xylene, dodecane, heptane, isooctane or cetylethylhexanoate. Preferred emollients used in the one embodiment are selected from mono- or diesters of linear and/or branched mono- and/or dicarboxylic acids having 2 to 44 C atoms with linear and/or branched saturated or unsaturated alcohols having 1 to 22 C atoms, the esterification products of aliphatic difunctional alcohols having 2 to 36 C atoms with monofunctional aliphatic carboxylic acids having 1 to 22 C atoms, long-chain aryl acid esters, such as e.g. esters of benzoic acid with linear and/or branched C6-C22-alcohols, or also benzoic acid isostearyl ester, benzoic acid butyloctyl ester or benzoic acid octyldodecyl ester, carbonates, preferably linear C6-C22-fatty alcohol carbonates, Guerbet carbonates, e.g. dicaprylyl carbonate, diethylhexyl carbonate, longer-chain triglycerides, i.e. triple esters of glycerol with three acid molecules, at least one of which is longer-chain, triglycerides based on C6-C10-fatty acids, linear or branched fatty alcohols, such as oleyl alcohol or octyldodecanol, and fatty alcohol ethers, such as dialykl ether e.g. dicaprylyl ether, silicone oils and waxes, e.g. polydimethylsiloxanes, cyclomethylsiloxanes, and aryl- or alkyl- or alkoxy-substituted polymethylsiloxanes or cyclomethylsiloxanes, Guerbet alcohols based on fatty alcohols having 6 to 18, preferably 8 to 10 carbon atoms, esters of linear C6-C22 fatty acids with linear C6-C22-fatty alcohols, esters of branched C6-C13-carboxylic acids with linear C6-C22-fatty alcohols, esters of linear C6-C22-fatty acids with branched C8-C18-alcohols, in particular 2-ethylhexanol or isononanol, esters of branched C6-C13-carboxylic acids with branched alcohols, in particular 2-ethylhexanol or isononanol, esters of linear and/or branched fatty acids with polyhydric alcohols (such as e.g. propylene glycol, dimer diol or trimer triol) and/or Guerbet alcohols, liquid mono-/di-/triglyceride mixtures based on C6-C18-fatty acids, esters of C6-C22-fatty alcohols and/or Guerbet alcohols with aromatic carboxylic acids, plant oils, branched primary alcohols, substituted cyclohexanes, ring-opening products of epoxidized fatty acid esters with polyols and/or silicone oils or a mixture of two or more of these compounds. The emollient used is preferably not miscible with water without phase separation.

Monoesters which are suitable as emollients and oil components are e.g. the methyl esters and isopropyl esters of fatty acids having 12 to 22 C atoms, such as e.g. methyl laurate, methyl stearate, methyl oleate, methyl erucate, isopropyl myristate, isopropyl palmitate, isopropyl stearate, isopropyl oleate. Other suitable monoesters are e.g. n-butyl stearate, n-hexyl laurate, n-decyl oleate, isooctyl stearate, isononyl palmitate, isononyl isononanoate, 2-ethylhexyl laurate, 2-ethylhexyl palmitate, 2-ethylhexyl stearate, 2-hexyldecyl stearate, 2-octyldodecyl palmitate, oleyl oleate, oleyl erucate, erucyl oleate and esters which are obtainable from technical-grade aliphatic alcohol cuts and technical-grade aliphatic carboxylic acid mixtures, e.g. esters of unsaturated fatty alcohols having 12 to 22 C atoms and saturated and unsaturated fatty acids having 12 to 22 C atoms, such as are accessible from animal and plant fats. However, naturally occurring monoester and wax ester mixtures such as are present e.g. in jojoba oil or in sperm oil are also suitable. Suitable dicarboxylic acid esters are e.g. di-n-butyl adipate, di-n-butyl sebacate, di-(2-ethylhexyl) adipate, di-(2-hexyldecyl) succinate, di-isotridecyl azelate. Suitable diol esters are e.g. ethylene glycol dioleate, ethylene glycol di-isotridecanoate, propylene glycol di-(2-ethylhexanoate), butanediol di-isostearate, butanediol di-caprylate/caprate and neopentyl glycol di-caprylate. Fatty acid triglycerides can be used; as such, for example, natural plant oils, e.g. olive oil, sunflower oil, soya oil, groundnut oil, rapeseed oil, almond oil, sesame oil, avocado oil, castor oil, cacao butter, palm oil, but also the liquid contents of coconut oil or of palm kernel oil, as well as animal oils, such as e.g. shark-fish liver oil, cod liver oil, whale oil, beef tallow and butter-fat, waxes, such as beeswax, carnauba palm wax, spermaceti, lanolin and neat's foot oil, the liquid contents of beef tallow or also synthetic triglycerides of caprylic/capric acid mixtures, triglycerides from technical-grade oleic acid, triglycerides with isostearic acid, or from palmitic acid/oleic acid mixtures, can be employed as emollients (oil phase). Ghe organic or oil-phase can be mineral oil or TEGOSOFT® M. Preferably, the emulsifier is chosen from polyoxyethylene sorbitan fatty acid esters, sucrose fatty acid esters, sorbitan fatty acid esters, polysorbates, polyvinyl alcohol, polyvinyl pyrrolidone, gelatin, lecithin, poly-oxyethylene castor oil derivatives, tocopherol, tocopheryl polyethylene glycol succinate, tocopherol palmitate and tocopherol acetate, polyoxyethylene-polyoxypropylene co-polymers, or their mixtures.

The microbeads of one embodiment give access to the compositions of one embodiment comprising these microbeads. The compositions of one embodiment may comprise at least one additional component chosen from the group of emollients, emulsifiers and surfactants, thickeners/viscosity regulators/stabilizers, UV light protection filters, antioxidants, hydrotropic agents (or polyols), solids and fillers, film-forming agents, insect repellents, preservatives, conditioning agents, perfumes, dyestuffs, biogenic active compounds, moisturizers and solvents. The additional components might be inside and/or outside the microbeads. Preferably the additional ingredients are present in the composition of one embodiment outside or within the microbeads.

The composition of one embodiment can be an emulsion, a suspension, a solution, a cream, an ointment, a paste, a gel, an oil, a powder, an aerosol, a stick or a spray. The microbeads or the compositions of one embodiment may be used as a transdermal drug delivery system/vehicle. When applied topically the microbeads congregate in wrinkles and folds of the skin.

In another aspect, a method of producing a hydrogel comprising hyaluronic acid, or salt thereof, crosslinked with divinylsulfone (DVS) by
(a) providing an alkaline solution of hyaluronic acid, or salt thereof;
(b) adding DVS to the solution of step (a), whereby the hyaluronic acid, or salt thereof, is crosslinked with the DVS to form a gel;
(c) treating the gel of step (b) with a buffer, wherein the gel swells and forms a hydrogel comprising hyaluronic acid, or salt thereof, crosslinked with DVS.

The hyaluronic acid, or salt thereof, has an average molecular weight of between 100 and 3,000 kDa, preferably between 500 and 2,000 kDa, and most preferably between 700 and 1,800 kDa. The initial concentration of hyaluronic acid, or a salt thereof, influences the properties of the resulting crosslinked gel, and of the swollen hydrogel. The alkaline solution comprises dissolved hyaluronic acid, or salt thereof, in a concentration of between 0.1%-40% (w/v). The pH value during the crosslinking reaction also influences the outcome, so in a preferred embodiment the invention relates to a method of the first aspect, wherein the alkaline solution comprises dissolved sodium hydroxide in a concentration of between 0.001-2.0 M. The concentration of the crosslinking agent can have a profound impact on the resulting gels. DVS is added to the solution of step (a) in a weight ratio of between 1:1 and 100:1 of HA/DVS (dry weight), preferably between 2:1 and 50:1 of HA/DVS (dry weight). An initial period of stirring during and/or immediately after adding the DVS to the HA-solution can be desirable to achieve satisfactory gelling. DVS is added with stirring to the solution of step (a), and wherein the solution temperature is maintained in the range of 5° C.-50° C., preferably in the range of 15° C.-40° C., more preferably in the range of 20° C.-30° C.; preferably the stirring is continued for a period of between 1-180 minutes. The DVS can be added without stirring to the solution of step (a).

The solution can be heated to a temperature in the range of 20° C.-100° C., preferably in the range of 25° C.-80° C., more preferably in the range of 30° C.-60° C., and most preferably in the range of 35° C.-55° C., and wherein the temperature is maintained in this range for a period of at least 5 minutes, preferably at least 10 minutes, 20 minutes, 30, 40, 50, 60, 70, 80, 90, 100, 110, 120, 130, 140, 150, 160, 170, or most preferably at least 180 minutes; preferably without stirring.

It is advantageous to leave the gel standing at room temperature for a brief period after the crosslinking reaction has taken place. The gel is maintained for a period of at least 5 minutes, preferably at least 10 minutes, 20 minutes, 30, 40, 50, 60, 70, 80, 90, 100, 110, 120, 130, 140, 150, 160, 170, or most preferably at least 180 minutes, at a temperature in the range of 0° C.-40° C., preferably in the range of 10° C.-30° C.

Many types of buffers, as are well known to the skilled person, have been envisioned as suitable for the swelling and neutralizing of the crosslinked gel of the invention. In a preferred embodiment the buffer comprises a buffer with a pH value in the range of 2.0-8.0, preferably in the range of 5.0-7.5. Optimally, a suitable buffer is chosen with a pH value, which results in that the swollen hydrogel has a pH value as close to neutral as possible. In a preferred embodiment, the buffer comprises a buffer with a pH value, which results in that the hydrogel has a pH value between 5.0 and 7.5. The buffer can be a phosphate buffer and/or a saline buffer. In the swelling step the buffer must have a sufficient volume for it to accommodate the swelling gel until the gel is fully swollen. The buffer in step (c) has a volume of at least 3 times the volume of the gel of step (b).

The swelling in step (c) is carried out at a temperature of between 20° C.-50° C. for a period of at least 5 minutes, preferably at least 10 minutes, 20 minutes, 30, 40, 50, 60, 70, 80, 90, 100, 110, 120, 130, 140, 150, 160, 170, or most preferably at least 180 minutes.

The hydrogel formed in step (c) can be washed at least once with water, water and a phosphate buffer, water and a saline buffer, or water and a phosphate buffer and a saline buffer, with a pH value in the range of 2.0-8.0, preferably in the range of 5.0-7.5.

### EXAMPLES

### Example 1

### Preparation of DVS Crosslinked Microparticles in Emulsion

This example illustrates the preparation of DVS-crosslinked microparticles. Sodium hyaluronate (HA, 580 kDa, 1.90 g) was dissolved in aqueous NaOH (0.2 M, 37.5 ml) by vigorous stirring at room temperature for 3 hours until a homogenous solution was obtained. Sodium chloride (0.29 g) was added and mixed shortly. Mineral oil (10.0 g) and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed for 1 min. to obtain a homogeneous distribution in the aq. phase. The water phase was then added within 2 minutes to the oil phase with mechanical stirring at low speed. An emulsion was formed immediately and stirring was continued for 30 minutes at room temperature. The emulsion was left over night at room temperature. The emulsion was neutralized to pH 7.0 by addition of aq. HCl (4 M, approx. 2.0 ml) and stirred for approx. 40 min.

### Example 2

### Preparation of DVS Crosslinked Microparticles in Emulsion Neutralized with Use of pH Indicator

This example illustrates the preparation of DVS-crosslinked microparticles with neutralization using a pH indicator. Sodium hyaluronate (HA, 580 kDa, 1.88 g) was dissolved in aqueous NaOH (0.2 M, 37.5 ml) by vigorous stirring at room temperature for 2 hours until a homogenous solution was obtained. Bromothymol blue pH indicator (equivalent range pH 6.6-6.8) was added (15 drops, blue color in solution). Sodium chloride (0.25 g) was added and mixed shortly.

Mineral oil (10.0 g) and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed very vigorously for 30 to 60 seconds to obtain a homogeneous distribution in the aq. phase. The water phase was then added within 30 sec. to the oil phase with mechanical stirring at 400 RPM. An emulsion was formed immediately and stirring was continued for 30 min. at room temperature. Neutralization was performed by addition of aq. HCl (4 M, 1.6 ml) and the emulsion was left at room temperature with magnetic stirring for 4 hours. The pH indicator present in the gel particles changed color to green. pH in the emulsion was measured by pH stick to 3-4. The emulsion was left in fridge over night. The pH indicator present in the gel particles had changed to yellow.

### Example 3

### Phase Separation of Emulsion, Swelling and Isolation of Microparticles

This example illustrates the breakage of the W/O emulsion followed by phase separation and dialysis. The crosslinked HA microparticles were separated from the W/O emulsion by organic solvent extraction. The W/O emulsion (5 g) and a mixture of n-butanol/chloroform (1/1 v %, 4.5 ml) was mixed vigorously by whirl mixing in a test tube at room temperature. Extra mQ-water (20 ml) was added to obtain phase separation. The test tube was centrifuged and three phases were obtained with the bottom phase being the organic phase, middle phase of gel particles and upper phase of clear aqueous solution. The top and bottom phases were discarded and the middle phase of gel particles was transferred into a dialysis tube (MWCO 12-14,000, Diameter 29 mm, Vol/Length 6.4 ml/cm). The sample was dialyzed overnight at room temperature in MilliQ®-water. The dialysate was changed two more times and left overnight. The resulting gel was thick and viscous and had swelled to a volume of approximately 50 ml, which correlated to 0.004 g HA/cm³.

### Example 4

### Preparation of DVS Crosslinked Microparticles in Emulsion and Separation of Microparticles

This example illustrates the preparation of DVS-crosslinked HA microparticles. Sodium hyaluronate (HA, 580 kDa, 1.89 g) was dissolved in aqueous NaOH (0.2 M, 37.5 ml). Sodium chloride (0.25 g) was added and the solution was stirred by magnetic stirring for 1 hour at room temperature until a homogeneous solution was obtained. TEGOSOFT® M (10.0 g) oil and ABIL® EM 90 surfactant (Cetyl PEG/PPG-10/1 Dimethicone, 1.0 g) were mixed by stirring.

Divinylsulfone (DVS, 320 microliter) was added to the aqueous alkaline HA-solution and mixed for 1 min. to obtain a homogenoues distribution in the aq. phase. The water phase was then added within 2 min. to the oil phase with mechanical stirring (300 RPM). An emulsion was formed immediately and stirring was continued for 30 min. at room temperature.

The emulsion was neutralized by addition of stociometric amounts of HCl (4 M, 1.8 ml) and stirred for approx. 40 min. The emulsion was broken by addition of a n-butanol/chloroform mixture (1:1 v %, 90 ml) and extra MilliQ®-water (100 ml) followed by magnetic stirring. The upper phase was separated in a volume of approx. 175 ml. The organic phase was mixed with mQ-water (30 ml) for a final washing. The combined water/gel phase (205 ml) were transferred to a dialysis tube (MWCO 12-14,000, Diameter 29 mm, Vol/Length 6.4 ml/cm) and dialysed against MilliQ®-water overnight at room temperature. The conductivity were decreased to 0.67 micro-Sievert/cm after subsequent change of water (3 times) and dialysis overnight (2 nights). The microparticles were assessed by microscopy (DIC 200×), see FIG. 1; the cross-section of one microparticle is indicated and labelled “21,587.92 nm”.

### Example 5

### Phase Separation of Emulsion and Isolation of Microparticles

This example illustrates the breakage of the W/O emulsion and isolation of the gel microparticles. The gel microparticles were separated from the W/O-emulsion by organic extractions. Examples of organic solvents which were used for this extraction were mixtures of butanol/chloroform in volume ratios (v %) of 75:20 to 20.80, respectively. The weight ratio (w %) of W/O emulsion to organic solvent was approximately 1:1.

Separation in small scale: The W/O emulsion (5 g) was weighed in centrifuge tubes (50 ml). A mixture of butanol/chloroform was prepared (1:1 v %) and from this mixture 4.5 ml was added (corresponds to 5 g) to the test tube. The test tube was carefully mixed to secure that all emulsion was dissolved. The test tube was mixed by Whirl mixing and left at room temperature for phase separation. Phase separation with water phase on top and organic phase at bottom with a white emulsion phase in between was often observed. Addition of more water and organic phases improved separation. The water phase was separated by decanting and further purified or characterized.

### Example 6

### Preparation of Water-in-Oil Emulsions

This example illustrates a composition in which the HA microparticles were formed.

A hot/cold procedure can be used with incorporation of a cold water phase B into a hot oil phase, which will shorten the time of manufacture. A non-limiting example of formulation could be as follows:

Phase A:
2.0% ABIL® EM 90 (cetyl PEG/PPG-10/1 dimethicone)
20.0% Mineral oil (or TEGOSOFT® M)

### Phase B:

0.5% Sodiumchloride
3.8% Hyaluronic acid
0.2 M NaOH (aq) up to 100%

### Phase C:

Approx. 0.6% Divinylsulfone

### Preparation:

1. Mix phase A at room temperature.
2. Phase B: Solubilize hyaluronic acid (Hyacare®) in aq. NaOH by stirring; then add NaCl and stir.
3. Add DVS to phase B and stir for 1 min.
4. Add phase B slowly to phase A with stirring.
5. Homogenise or stir for a short time and leave to react.
6. Stirring and swelling.
7. Continue stirring below 30° C.
8. Neutralize.

### Example 7

### Preparation and Separation of DVS Cross-Linked Microparticles

Sodium hyaluronate (HA, 580 kDa, 1.88 g) was dissolved in aqueous NaOH (0.2 M, 37.5 mL). Sodium chloride (0.25 g) was added and the solution was stirred by magnetic stirring for 1 hour at room temperature until a homogeneous solution was obtained. The oil: TEGOSOFT® M (10.0 g) and surfactant: ABIL® EM 90 (Cetyl PEG/PPG-10/1 Dimethicone, 1.0 g) was mixed by stirring. Divinylsulfone (DVS, 320 microliter) was added to the aqueous alkaline HA-solution and mixed for 1 min to obtain a homogenoues distribution in the aq. phase. The water phase was then added within 2 min to the oil phase with mechanical stirring (300 RPM). An emulsion was formed immediately and stirring was continued for 30 min at room temperature.

The emulsion was neutralized by addition of stociometric amounts of HCl (4 M, 1.8 mL) and stirred for approx. 40 min. The emulsion was transferred to a separation funnel, and broken by addition of a n-butanol/chloroform mixture (1:1 v %, 90 mL) and extra millliQ™-water (100 mL) followed by vigorous shaking. The upper phase was separated in a volume of approx. 175 mL. The organic phase was washed with millliQ™-water (100 mL). The combined water/gel phase was transferred to a dialysis tube (MWCO 12-14,000, Diameter 29 mm, Vol/Length 6.4 mL/cm) and dialysed against millliQ™-water overnight at room temperature. The conductivity was decreased to 10 micro-Sievert/cm after subsequent change of water (3 times) and dialysis overnight (2 nights). The microparticles were assessed by microscopy (FIG. 4).

### Example 8

### Washing Procedure to Purify Microparticles

This example illustrates the final isolation and purification of the microparticles.

100 mL particles previously isolated were re-suspended in a Na2HPO4/NaH2PO4buffer (0.15 M, 400 mL), and stirred slowly for ½ hour. The suspension stood at 5° C. for 2 hours and solidified oil droplets were removed. The solution was then filtered through a mesh and washed further with 2×50 mL buffer. Particles were allowed to drip-dry, before characterization (FIG. 5).

### Example 9

### Investigation of Rheological Properties of Microparticles

This example illustrates performance of rheological studies on particles. A particle sample is analyzed on an Anton Paar rheometer (Anton Paar GmbH, Graz, Austria, Physica MCR 301, Software: Rheoplus), by use of a 50 mm 2° cone/plate geometry. First the linear range of the visco-elastic properties G′ (Storage modulus) and G″ (Loss modulus) of the material is determined by an amplitude sweep with variable strain, γ. Secondary a Frequency sweep is made, and based on values of the visco-elastic values, G′ and G″, tan δ can be calculated as a value for week/strong gel behaviors.

### Example 10

### Investigation of Syringeability Experiments on Texture Analyzer

This example illustrates performance of an investigation of force applied to inject at a certain speed, as a function of the homogeneity of the sample. A particle sample is transferred to a syringe applied with a needle, either 27G×½″, 30G×½″, and is set in a sample rig, in a texture analyzer (Stable Micro Systems, Surrey, UK, TA.XT Plus, SoftWare: Texture Component 32). The test is performed with an injection speed at 12.5 mm/min., over a given distance.

### Example 11

### Preparation of DVS-Crosslinked HA Hydrogels

This example illustrates the preparation of DVS-cross-linked HA hydrogels with concomitant swelling and pH adjustment.

Sodium hyaluronate (HA, 770 kDa, 1 g) was dissolved into 0.2M NaOH to give a 4% (w/v) solution, which was stirred at room temperature, i.e. about 20° C., for 1 h. Three replicates were prepared. Divinylsulfone (DVS) was then added to the HA solutions in sufficient amount to give HA/DVS weight ratios of 10:1, 7:1, and 5:1, respectively. The mixtures were stirred at room temperature for 5 min and then allowed to stand at room temperature for 1 h. The gels were then swollen in 160 mL phosphate buffer (pH 4.5 or 6.5) for 24 h, as indicated in Table 1.

TABLE 1
Conditions for DVS-HA hydrogel preparation.

| | HA/DVS | |
|---|---|---|
| | weight | Phosphate buffer |
| Gel ID | ratio | used for swelling |
| 1 | 5:1 | 160 ml (pH 4.5) |
| 2 | 7:1 | 80 ml (pH 4.5) + 80 |
| | | ml (pH 6.5) |
| 3 | 10:1 | 160 ml (pH 6.5) |

The pH of the gels was stabilized during the swelling step. After swelling, any excess buffer was removed by filtration and the hydrogels were briefly homogenized with an IKA® ULTRA-TURRAX® T25 homogenizer (Ika Labortechnik, DE). The volume and pH of the gels were measured (see Table 2).

TABLE 2
Characteristics of DVS-HA hydrogels.

| | HA/DVS | Volume of | HA | | | |
|---|---|---|---|---|---|---|
| Gel | weight | swollen | concentration | | | Soft- |
| ID | ratio | gel | (w/v) | pH | Appearance | ness |
| 1 | 5:1 | 70 mL | 1.4% | 7.1 | Transparent, | + |
| | | | | | homogenous | |
| 2 | 7:1 | 70 mL | 1.4% | 7.6 | Transparent, | ++ |
| | | | | | homogenous | |
| 3 | 10:1 | 70 mL | 1.4% | 7.5 | Transparent, | +++ |
| | | | | | homogenous | |

The pH of the hydrogels ranged from 7.1 to 7.6 (table 2), which confirms that the swelling step can be utilized to adjust the pH in this process. All the hydrogels occupied a volume of 70 mL, which corresponds to a HA concentration of ca. 1.4% (w/v). They were transparent, coherent and homogenous. Softness increased with decreasing cross-linking degree (Table 2).

### Example 12

### Preparation of Homogenous DVS-Crosslinked HA Hydrogels

This example illustrates the preparation of highly homogenous DVS-cross-linked HA hydrogels.

Sodium hyaluronate (770 kDa, 2 g) was dissolved into 0.2M NaOH with stirring for approx. 1 hour at room temperature to give a 8% (w/v) solution. DVS was then added so that the HA/DVS weight ratio was 7:1. After stirring at room temperature for 5 min, one of the samples was heat treated at 50° C. for 2 h without stirring, and then allowed to stand at room temperature overnight. The resulting cross-linked gel was swollen into 200 ml phosphate buffer (pH 5.5) 37° C. for 42 or 55 h, and finally washed twice with 100 ml water, which was discarded. Volume and pH were measured, as well as the pressure force necessary to push the gels through a 27G*½ injection needle (see Table 3).

TABLE 3
Characteristics of DVS-cross-linked HA hydrogels.

| | | | | | | | Stability |
|---|---|---|---|---|---|---|---|
| | | | | | | | of |
| | | Volume | HA | | | | pressure |
| | | of | concen- | | | | force |
| Gel | Heat | swollen | tration | | | | during |
| ID | treated | gel | (w/v) | pH | Appearance | Softness | injection |
| 1 | Yes | 145 mL | 1.4% | 6.1 | Transparent, | +++ | +++ |
| | | | | | homogenous | | |
| 2 | No | 90 mL | 1.1% | 6.7 | Transparent, | + | + |
| | | | | | homogenous | | |

The cross-linked HA hydrogel prepared according to this example exhibited a higher swelling ratio and an increased softness compared to a control hydrogel which was not heat treated (Table 3). The pressure force applied during injection through a 27G*½ needle was more stable than that of the latter sample, indicating that the cross-linked HA hydrogel is more homogenous.

### Example 13

### Biostability of DVS-Crosslinked HA Hydrogels

This example illustrates the in vitro biostability of DVS-cross-linked HA hydrogels using enzymatic degradation.

A bovine testes hyaluronidase (HAase) solution (100 U/mL) was prepared in 30 mM citric acid, 150 mM Na₂HPO₄, and 150 mM NaCl (pH 6.3). DVS-HA cross-linked hydrogel samples (ca. 1 mL) were placed into safe-lock glass vials, freeze-dried, and weighed (W₀; Formula 1). The enzyme solution (4 mL, 400 U) was then added to each sample and the vials were incubated at 37° C. under gentle shaking (100-200 rpm). At predetermined time intervals, the supernatant was removed and the samples were washed thoroughly with distilled water to remove residual salts, they were then freeze-dried, and finally weighed (Wₜ; Formula 1).

The biodegradation is expressed as the ratio of weight loss to the initial weight of the sample (Formula 1). Weight loss was calculated from the decrease of weight of each sample before and after the enzymatic degradation test. Each biodegradation experiment was repeated three times. DVS-HA hydrogels prepared as described in example 2 (‘Heated’) were compared to DVS-HA hydrogels which had not been heat treated (‘Not heated’). For both types of gel, degradation was fast during the first four hours, and then proceeded slower until completion at 24 h. Importantly there was a significant variation of the weight loss values for the samples which had not been heated as compared to the hydrogel prepared with a heating step as described in example 2. This clearly illustrates that a highly homogenous DVS-cross-linked HA hydrogel is obtained by using the process described in example 2.

### Example 14

### Preparation of Water-in-Oil Emulsions for Cosmetics

In this and in the following example, DVS-crosslinked HA hydrogels were formulated into creams and serums, that when applied to the skin increase the skin moisturization and elasticity, and provide immediate anti-aging effect, as well as film-forming effect

A typical formulation of a water-in-oil (w/o) emulsion containing 2% DVS-cross-linked HA. Each phase (A to E) was prepared separately by mixing the defined ingredients (see Table 4). Phase B was then added to phase A under stirring with a mechanical propel stirring device and at a temperature less than 40° C. Phase C was then added followed by phase D and finally phase E under stirring. Formulations were also made, wherein the HA hydrogel concentration was 4%, 6% and 8%, respectively, in Phase D, to give a range of w/o formulations.

TABLE 4

| | | Proportion | |
|---|---|---|---|
| Phase | Ingredient | (w/w) | Function |
| A | Cyclopentasiloxane, dimethicone | 10% | Emollient |
| | Cyclopentasiloxane | 15% | Emollient |
| | Cyclopentasiloxane and PEG/PPG- | 4% | Emulsifier |
| | 20/15 Dimethicone | | |
| | Hydrogenated polydecene | 8% | Emollient |
| B | Water | 49.3% | |
| | Sodium chloride | 0.2% | |
| C | Tocopheryl acetate | 0.5% | Antioxidant |
| D | DVS Cross-linked sodium | 2% | |
| | hyaluronate | | |
| | Water | 10% | |
| E | Phenoxyethanol, ethylhexylglycerin | 1% | Preservative |

Another typical formulation of a w/o-emulsion containing 2% DVS-crosslinked HA is shown in table 5. Each phase (A to F) in table 5 was prepared separately by mixing the defined ingredients (see Table 5). Phase B was mixed with phase A and the resulting oil phase was heated at 75° C. Phase C was also heated to 75° C. The oil phase was added to phase C at 75° C. under stirring with a mechanical propel stirring device. The emulsion was then cooled down to less than 40° C., after which phase D was added, followed by phase E and finally phase F under stirring. Formulations were also made, wherein the HA hydrogel concentration was 4%, 6% and 8%, respectively, in Phase E, to give a range of w/o formulations.

TABLE 5

| | | Proportion | |
|---|---|---|---|
| Phase | Ingredient | (w/w) | Function |
| A | Hydrogenated polydecene | 18% | Emollient |
| | Acrylates/C10-30 alkyl acrylate | 1% | Thickener |
| | crosspolymer | | |
| B | Sodium cocoyl Glutamate | 10% | Emulsifier |
| C | Aqua | 53.5% | |
| | Distarch Phosphate | 2% | Texture agent |
| D | Tocopheryl acetate | 0.5% | Antioxidant |
| | Cyclopentasiloxane, dimethicone | 2% | Feeling and |
| | | | spreading agent |
| E | Cross-linked sodium | 2% | |
| | hyaluronate | | |
| | Aqua | 10% | |
| F | Phenoxyethanol, ethylhexylglycerin | 1% | Preservative |

### Example 15

### Preparation of Silicone Serums

A typical formulation of a silicone serum containing 2% DVS-cross-linked HA was prepared as shown in table 6. All ingredients were mixed at the same time under very high stirring and at less than 40° C. (see table 6). Formulations were also prepared, wherein the HA hydrogel concentration was 4%, 6% and 8%, respectively, to give a range of serums.

TABLE 6

| | Proportion | |
|---|---|---|
| Ingredient | (w/w) | Function |
| Cyclopentasiloxane | 60% | Line blurring effect, |
| C30-45 Alkyl Cetearyl Dimethicone | | thickener, vehicle |
| Crosspolymer | | |
| Cyclopentasiloxane | 34.5% | Vehicle, emollient |
| Polymethylsilsesquioxane | 2.5% | Soft powdery feel |
| Cross-linked sodium | 2% | |
| hyaluronate | | |
| Phenoxyethanol, ethylhexylglycerin | 1% | Preservative |

### Example 16

### pH Equilibration During Swelling; a Kinetics Study

A kinetics study showed that DVS cross-linked HA hydrogels with neutral pH are obtained after swelling in phosphate buffer (pH 7.0) for 8 to 14 hours, depending on the degree of cross-linking A set of DVS cross-linked HA hydrogels was prepared as described in the above, using from 4 to 8% HA solution, and using various amounts of DVS cross-linker, as indicated in Table 7.

TABLE 7
Initial HA concentration HA/DVS weight

| Entry | (w/v) | ratio |
|---|---|---|
| 1 | 4% | 2.5:1 |
| 2 | 6% | 15:1 |
| 3 | 8% | 15:1 |
| 4 | 6% | 10:1 |

At regular intervals (every 2 hours), the hydrogels were removed during the heat-treatment and decanted, and pH was measured (see FIG. 2). Fresh swelling buffer was used after each measurement. The results show that, for all hydrogels, pH ranged between 11 and 12 after 2-hours of swelling. Then pH gradually decreased to 7.2-7.5.

The decrease was faster for the hydrogels that were less cross-linked, i.e., where the HA/DVS-ratio was higher. The decrease in pH is shown for the HA 6% solution and two different ratios of HA/DVS in FIG. 2, where the HA/DVS ratio of 10:1 is labelled with triangles, and 15:1 is labelled with squares. In these two cases, pH was neutralized within 8 hours. In contrast, neutral pH was reached after 14 hour-swelling for hydrogels with either a higher HA concentration (e.g. 8%) or a higher degree of cross-linking (e.g. HA/DVS ratio of 2.5). These observations are in accordance with the fact that HA molecules in the low cross-linked hydrogels exhibit greater freedom and flexibility, allowing good hydration and thereby faster pH equilibration.

### Example 17

### Visco Elastic Properties of Hydrogels Based on DVS-Crosslinked HA

The rheological measurements were performed on a Physica MCR 301 rheometer (Anton Paar, Ostfildern, Germany) using a plate-plate geometry and at a controlled temperature of 25° C. The visco-elastic behavior of the samples was investigated by dynamic amplitude shear oscillatory tests, in which the material was subjected to a sinusoidal shear strain. First, strain/amplitude sweep experiments were performed to evaluate the region of deformation in which the linear viscoelasticity is valid. The strain typically ranged from 0.01 to 200% and the frequency was set to 1 Hz. Then, in the linear visco-elastic regions, the shear storage modulus (or elastic modulus G′) and the shear loss modulus (or viscous modulus, G″) values were recorded from frequency sweep experiments at a constant shear strain (10%) and at a frequency between 0.1 and 10 Hz. The geometry, the NF and the gap were PP 25, 2 and 1 mm, respectively.

G′ gives information about the elasticity or the energy stored in the material during deformation, whereas G″ describes the viscous character or the energy dissipated as heat. In particular, the elastic modulus gives information about the capability of the sample to sustain load and return in the initial configuration after an imposed stress or deformation. In all experiments, each sample was measured at least three times.

In case of the hydrogel with a higher degree of cross-linking (i.e. lower HA/DVS ratio: 10/1) G′ is one order of magnitude higher than G″, indicating that this sample behaves as a strong gel material. Briefly, the overall rheological response is due to the contributions of physical and chemical crosslinks, and to topological interactions among the HA macromolecules. The interactions among the chains bring about a reduction of their intrinsic mobility that is not able to release stress, and consequently the material behaves as a three-dimensional network, where the principal mode of accommodation of the applied stress is by network deformation. Moreover, this hydrogel was more elastic than that with a lower degree of cross-linking (i.e., higher ratio of HA/DVS: 15:1). Indeed, the higher the number of permanent covalent cross-links, the larger the number of entanglements, and therefore the higher the elastic response of the hydrogel.

### Example 18

### Crosslinked HA/DVS Hydrogel with Preservative

A DVS-cross-linked HA hydrogel was prepared using 1.5 g of sodium HA in 0.2 M NaOH to give a 6% (w/v) solution. The HA/DVS weight ratio was 10:1. The hydrogel was prepared in three replicates according to the procedure described in example 2 until the swelling step, after which it was treated as follows: After incubation in an oven at 50° C. for two hours, the hydrogel was immersed into Na2HPO4/NaH2PO4 buffer (1 L, 50 mM, pH 7.0) containing the preservative (2-phenoxyethanol/3[(2-ethylhexyl)oxy]1,2-propanediol).

The concentration of preservative was 10 mL/mL to target a final concentration of 1% (v/v) in the swollen hydrogel. It was anticipated that the preservative would diffuse into the hydrogel during the incubation, and that at the same time, microbial contamination in the buffer would be prevented.

The vessel was covered with parafilm and placed in an oven at 37° C. After 1 h, the swelling bath was removed and the hydrogel was swollen in a fresh phosphate buffer containing 10 mL/mL preservative for 6-7 h. This step was repeated until the swelling time was 12 h, whereafter the pH was measured. Swelling was continued for another 2.5 h to reach neutral pH.

The amount of preservative incorporated into the hydrogel was determined by UV-spectrophotometry (Thermo Electron, Nicolet, Evolution 900, equipment nr. 246-90). A 1% (v/v) solution of the preservative in phosphate buffer was first analyzed to select the wavelength. Approximately 5 mL of hydrogel were collected using a pipette. Typically, samples were collected in the center of the swollen round hydrogel, and in the north, east, south, and west “sides” of the round gel.

The samples were then transferred into a cuvette and the absorbance was read at 292 nm. Each sample was read three times and the absorbance was zeroed against a blank DVS-cross-linked HA hydrogel, containing no preservative.

The results showed that the amount of preservative incorporated in the DVS-HA hydrogel ranged between 0.91% and 1.02% (see Table 10). There was very good reproducibility between the replicates. Importantly, no significant difference between samples from the same hydrogel was observed, indicating a homogenous diffusion of the preservative into the hydrogel.

TABLE 8
Amount of incorporated preservative into DVS-HA
hydrogel upon swelling in a 1% preservative-
spiked phosphate buffer for 14.5 h.

| | | | Preservative | Average |
|---|---|---|---|---|
| | Sample | Absorbance* | concentration | concentration |
| Sample ID | site | (292 nm) | (%, v/v) | (%, v/v) |
| Replicate | Center | 0.072 | 1.02 | 0.91 |
| 1 | Side | 0.058 | 0.82 | |
| | Side | 0.066 | 0.94 | |
| | Side | 0.057 | 0.81 | |
| | Side | 0.068 | 0.97 | |
| Replicate | Middle | 0.076 | 1.08 | 1.02 |
| 2 | Side | 0.069 | 0.98 | |
| | Side | 0.082 | 1.17 | |
| | Side | 0.071 | 1.01 | |
| | Side | 0.062 | 0.88 | |
| Replicate | Middle | 0.083 | 1.18 | 1.02 |
| 3 | Side | 0.074 | 1.05 | |
| | Side | 0.069 | 0.98 | |
| | Side | 0.066 | 0.94 | |
| | Side | 0.068 | 0.97 | |

### Example 19

### Biodegradable Polymer Choices

The time of degradation may be adjusted based on the polymer mixture in Table 1 below. Examples 1 and 2 below are examples of matrix incorporation of drug or drugs into a biodegradable polymer to control the releases the drugs.

TABLE 1
Biodegradation Time and Composition

| Polymer | |
|---|---|
| (mos) | Degradation Time |
| 50:50 DL-PLG | 1-2 |
| 65:35 DL-PLG | 3-4 |
| 75:25 DL-PLG | 4-5 |
| 85:15 DL-PLG | 5-6 |
| DL-PLA | 12-16 |
| L-PLA | >24 |
| PGA | 6-12 |
| PCL | >24 |

Different types of biodegradable polymer may be used to control the degradation timing and/or to control the degradation by-products. Some biodegradable polymers are:
PGA, PLA and their copolymers are some of the most frequently used biodegradable polymer materials in part because their properties that can be tuned by changing the polymer composition within the basic PLA/PGA theme.
Poly(glycolic acid) (PGA) is very susceptible to hydrolysis
Poly(lactic acid) (PLA) exists in D and/or L enantiomer mixtures of these results in varying biodegradation timing due to crystalline regions that form when they are in mixture which limits the level of hydrolysis possible

Polydioxanone (PDS)
Poly(ε-caprolactone)
Poly(DL-lactide-co-ε-caprolactone)

### Surfactant Choices:

The particle sizes of the micro capsules are directly controlled by the interfacial chemistry of the organic phase and the aqueous phase. A surfactant is often used to mediate interfacial surface chemistry between an oily substance and the aqueous environment. A surfactant is a detergent that is in an aqueous solution. Surfactants are large molecules that have both polar and non-polar ends. The polar end of the molecule will attach itself to water, also a polar molecule. The non-polar end of the molecule will attract NAPL (non-aqueous phase liquid) compounds.

Examples of surfactants that are used for solubilization are:

1. Sioponic 25-9 which is a linear alcohol ethoxylate, and has a solubilization value of 2.75 g/g
2. Tergitol which is an ethylene oxide/propylene oxide with a solubilization value of 1.21 g/g
3. Tergitol XL-80N which is an ethylene oxide propylene oxide alkoxylate of primary alcohol with a solubilization value of 1.022 g/g
4. Tergitol N-10 which is an a trimethyl nonal ethoxylate with a solubilization value of 0.964 g/g
5. Rexophos 25/97 which is a phosphated nonylphenol ethooxylate with a solubilization value of 0.951 g/g

### Example 20

### Biodegradable Micro Particles Containing Anti-Inflammatory, Cortical Steroid or Steroids

a. Delayed 30 days
b. Controlled release over 120 days

### Organic Phase:

Make a 20% DLPLG polymer with methylene chloride
The DLPLA polymer contains 65% DL and 35% PLG
Weigh 0.02 g triamcinolone into a glass vial
Dispense 2 mL of 20% DLPLG polymer solution into the vial containing the triamcinolone
Dissolve the drug completely using an orbital mixer

### Aqueous Phase:

Make 100 mL of SDS (sodium dodecyl sulfate) at a 0.1 molar concentration in DI water
Dispense 8 mL of SDS 0.1 molar solution into the drug/polymer solution

### Solvent Evaporation:

Place the glass vial containing the reaction mixture under the impeller mixer.
Turn the mixer up to 1200 rpm.
Unless the speed required to produce a desired particle size is known, start slowly and work up to an impeller speed that produces the desired particle size.
After the speed to produce the desired particle size has been figured out. Begin heating the vessel in a 80 C water bath with continuous mixing
When all the methylene chloride in the organic phase has been boiled off, this case, the time is 45 minutes, stop heating
Continue mixing, let reaction cool to room temperature slowly
The rate of cooling and mixing effect the agglomeration of the particles to each other
The SDS may be washed by continuously exchanging the solution mixture with DI water
Collect the particles by filtration
Dry the particles at 80 C in a vacuum oven

### Fluidized Bed Encapsulation

Make a 3% and 5% polymer composition 50:50 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drug

### Example 21

### Biodegradable Microcapsule Containing Anti-Proliferative Pharmaceutical

a. Delayed 60 days
b. Controlled release over 365 days

### Organic Phase:

Make a 20% DLPLG polymer with methylene chloride
The DLPLA polymer contains 100% PGA
Weigh 0.02 g sirolimus into a glass vial
Dispense 2 mL of 20% DLPLG polymer solution into the vial containing the triamcinolone
Dissolve the drug completely using an orbital mixer

### Aqueous Phase:

Make 100 mL of SDS (sodium dodecyl sulfate) at a 0.1 molar concentration in DI water
Dispense 8 mL of SDS 0.1 molar solution into the drug/polymer solution

### Solvent Evaporation:

Place the glass vial containing the reaction mixture under the impeller mixer.
Turn the mixer up to 1200 rpm.
Unless the speed required to produce a desired particle size is known, start slowly and work up to an impeller speed that produces the desired particle size.
After the speed to produce the desired particle size has been figured out. Begin heating the vessel in a 80 C water bath with continuous mixing
When all the methylene chloride in the organic phase has been boiled off, this case, the time is 45 minutes, stop heating

Continue mixing, let reaction cool to room temperature slowly
The rate of cooling and mixing effect the agglomeration of the particles to each other
The SDS may be washed by continuously exchanging the solution mixture with DI water
Collect the particles by filtration
Dry the particles at 80 C in a vacuum oven

### Fluidized Bed Encapsulation

Make a 3% and 5% polymer composition 65:35 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drug

### Example 22

### Dermal Filler Composition Containing Anesthetic, Cortical Steroid and Anti-Proliferative Pharmaceutical

a. Biodegradable microcapsule containing a cortical steroid delayed 30 days, controlled release over 120 days
b. Biodegradable microcapsule containing an anti-proliferative pharmaceutical delayed 60 days, controlled released over 365 days

### Composition Mixture (Dry)

Hyaluronic acid, cross-linked 60%-95%
Anti-inflammatory drug containing micro particles 5%-20%
Antiproliferative drug containing micro particles 5%-20%
Anesthetic drug (lidocaine hydrochloride) 0.1%-5%

Reconstitute in phosphate buffered saline at 0.024 g/mL concentration

### Example 23

### Encapsulation of an Anti-Proliferative Pharmaceutical a Biodegradable Acrylic Acid Copolymer

### Shell Formation Phase

Dissolve the following, which makes up the organic phase:
0.25 g of a biodegradable acrylic acid copolymer in
0.75 g of sirolimus
2 mL methylene chloride
0.1 mL ethanol
Aqueous phase is:
75 mL of 0.5% polyvinyl alcohol solution maintained at room temperature
Disperse the two phases using a mechanical mixer at 1200 rpm or whichever speed that gives the desire particle size
Add an appropriate amount of amine or in this case triethyl amine

Continue mixing for 2 hours with reaction vessel in a water bath at 80 C
Add 0.1 mL of Jeffamine (T-403) to harden the capsule surface
Continue mixing, let reaction cool to room temperature slowly
The rate of cooling and mixing effect the agglomeration of the particles to each other
The polyvinyl alcohol may be washed by continuously exchanging the solution mixture with fresh DI water
Collect the particles by filtration
Dry the particles at 80 C in a vacuum oven

### Fluidized Bed Encapsulation

Make a 3% and 5% polymer composition 65:35 PL:PLG in methylene chloride
Put the dried particle containing drug into the fluidized bed
Deposit a uniform layer of polymer onto the drug containing particles using the 5% polymer solution. Adjust the spray rate and air flow to get an optimized particle bed.
Use the 3% polymer solution to finalized the process ensuring that there are no pin holes to eventual cause unwanted early release of the drugIn addition to biocompatibility, the other important characteristics of the gel slurries according to the one embodiment which determine their usefulness in various medical fields is the complex combination of their rheological properties. These properties include viscosity and its dependence on shear rate, the ratio between elastic and viscous properties in dynamic mode, relaxation behavior and some others which are discussed below in more detail. In general, the rheology of the products of the one embodiment can be controlled over very broad limits, essentially by two methods. According to the first such method, the rheological properties of each of the two phases forming the viscoelastic gel slurry are controlled in such a way that gives the desirable rheology for the final product. The second such method of controlling the rheology of the gel slurry consists of selecting a proper ratio for two phases. But because these parameters, i.e. rheology of the two phases and their ratio determine some other important properties of the products of one embodiment, the best way to control the rheology should be selected ad hoc for each specific case.

The gels suitable for the use in the products according to the one embodiment can represent very many different kinds of rheological bodies varying from hard fragile gels to very soft deformable fluid-like gels. Usually, for the gels which are formed without a crosslinking reaction, for example, a conventional gelatin gel, the hardness and elasticity of the gel increases with increasing polymer concentration. The rheological properties of a crosslinked gel are usually a function of several parameters such as crosslinking density, polymer concentration in the gel, composition of the solvent in which the crosslinked polymer is swollen. Gels with different rheological properties based on hyaluronan and hylan are described in the above noted U.S. Pat. Nos. 4,605,691, 4,582,865 and 4,713,448. According to these patents, the rheological properties of the gel can be controlled, mainly, by changing the polymer concentration in the starting reaction mixture and the ratio of the polymer and the crosslinking agent, vinyl sulfone. These two parameters determine the equilibrium swelling ratio of the resulting gel and, hence, the polymer concentration in the final product and its rheological properties.

A substantial amount of solvent can be removed from a gel which had previously been allowed to swell to equilibrium, by mechanical compression of the gel. The compression can be achieved by applying pressure to the gel in a closed vessel with a screen which is permeable to the solvent and impermeable to the gel. The pressure can be applied to the gel directly by means of any suitable device or through a gas layer, conveniently through the air. The other way of compressing the gel is by applying centrifugal force to the gel in a vessel which has at its bottom the above mentioned semipermeable membrane. The compressibility of a polymeric gel slurry depends on many factors among which are the chemical nature of the gel, size of the gel particles, polymer concentration and the presence of a free solvent in the gel slurry. In general, when a gel slurry is subjected to pressure the removal of any free solvent present in the slurry proceeds fast and is followed by a much slower removal of the solvent from the gel particles. The kinetics of solvent removal from a gel slurry depends on such parameters as pressure, temperature, configuration of the apparatus, size of the gel particles, and starting polymer concentration in the gel. Usually, an increase in pressure, temperature, and filtering surface area and a decrease in the gel particle size and the initial polymer concentration results in an increase in the rate of solvent removal.

Partial removal of the solvent from a gel slurry makes the slurry more coherent and substantially changes the rheological properties of the slurry. The magnitude of the changes strongly depends on the degree of compression, hereinafter defined as the ratio of the initial volume of the slurry to the volume of the compressed material.

The achievable degree of compression, i.e. compressibility of a gel slurry, is different for different gels. For hylan gel slurries in saline, for example, it is easy to have a degree of compression of 20 and higher.

Reconstitution of the compressed gel with the same solvent to the original polymer concentration produces a gel identical to the original one. This has been proven by measuring the rheological properties and by the kinetics of solvent removal from the gel by centrifuging.

It should be understood that the polymer concentration in the gel phase of the viscoelastic mixtures according to the one embodiment may vary over broad ranges depending on the desired properties of the mixtures which, in turn, are determined by the final use of the mixture. In general, however, the polymer concentration in the gel phase can be from 0.01 to 30%, preferably, from 0.05 to 20%. In the case of hylan and hyaluronan pure or mixed gels, the polymer concentration in the gel is preferably, in the range of 0.1 to 10%, and more preferably, from 0.15 to 5% when the swelling solvent is physiological saline solution (0.15M aqueous sodium chloride).

As mentioned above the choice of a soluble polymer or polymers for the second phase of the viscoelastic gel slurries according to one embodiment is governed by many considerations determined by the final use of the product. The polymer concentration in the soluble polymer phase may vary over broad limits depending on the desired properties of the final mixture and the properties of the gel phase. If the rheological properties of the viscoelastic gel slurry are of prime concern then the concentration of the soluble polymer may be chosen accordingly with due account taken of the chemical nature of the polymer, or polymers, and its molecular weight. In general, the polymer concentration in the soluble phase may be from 0.01% to 70%, preferably from 0.02 to 40%. In the case when hylan or hyaluronan are used as the soluble polymers, their concentration may be in the range of 0.01 to 10%, preferably 0.02 to 5%. In the case where other glycosaminoglycans such as chondroitin sulfate, dermatan sulfate, etc., are used as the soluble polymers, their concentration can be substantially higher because they have a much lower molecular weight.

The two phases forming the viscoelastic gel slurries according to one embodiment can be mixed together by any conventional means such as any type of stirrer or mixer. The mixing should be long enough in order to achieve uniform distribution of the gel phase in the polymer solution. As mentioned above, the gel phase may already be a slurry obtained by disintegrating a gel by any conventional means such as pushing it through a mesh or a plate with openings under pressure, or by stirring at high speed with any suitable stirrer. Alternatively, the viscoelastic mixed gel slurries can be prepared by mixing large pieces of gel with the polymer solution and subsequently disintegrating the mixture with formation of the viscoelastic slurry by any conventional means discussed above. When the first method of preparing a mixed gel slurry according to one embodiment is used, the gel slurry phase can be made of a gel swollen to equilibrium, and in this case there is no free solvent between the gel particles, or it may have some free solvent between gel particles. In the latter case this free solvent will dilute the polymer solution used as the second phase. The third type of gel slurry used as the gel phase in the mixture is a compressed gel whose properties were discussed above. When a compressed gel slurry is mixed with a polymer solution in some cases the solvent from the solution phase will go into the gel phase and cause additional swelling of the gel phase to equilibrium when the thermodynamics of the components and their mixture allows this to occur.

The composition of the viscoelastic mixed gel slurries according to one embodiment can vary within broad limits. The polymer solution in the mixture can constitute from 0.1 to 99.5%, preferably, from 0.5 to 99%, more preferably, from 1 to 95%, the rest being the gel phase. The choice of the proper composition of the mixture depends on the properties and composition of the two components and is governed by the desirable properties of the slurry and its final use.

The viscoelastic gel mixtures according to one embodiment, in addition to the two major components, namely, the polymeric gel slurry and the polymer solution, may contain many other components such as various physiologically active substances, including drugs, fillers such as microcrystalline cellulose, metallic powders, insoluble inorganic salts, dyes, surface active substances, oils, viscosity modifiers, stabilizers, etc., all depending upon the ultimate use of the products.

The viscoelastic gel slurries according to one embodiment represent, essentially, a continuous polymer solution matrix in which discrete viscoelastic gel particles of regular or irregular shape are uniformly distributed and behave rheologically as fluids, in other words, they exhibit certain viscosity, elasticity and plasticity. By varying the compositional parameters of the slurry, namely the polymer concentration in the gel and the solution phases, and the ratio between two phases, one may conveniently control the rheological properties of the slurry such as the viscosity at a steady flow, elasticity in dynamic mode, relaxation properties, ratio between viscous and elastic behavior, etc.

The other group of properties which are strongly affected by the compositional parameters of the viscoelastic gel slurries according to one embodiment relates to diffusion of various substances into the slurry and from the slurry into the surrounding environment. The diffusion processes are of great importance for some specific applications of the viscoelastic gel slurries in the medical field such as prevention of adhesion formation between tissues and drug delivery as is discussed below in more detail.

It is well known that adhesion formation between tissues is one of the most common and extremely undesirable complications after almost any kind of surgery. The mechanism of adhesion formation normally involves the formation of a fibrin clot which eventually transforms into scar tissue connecting two different tissues which normally should be separated. The adhesion causes numerous undesirable symptoms such as discomfort or pain, and may in certain cases create a life threatening situation. Quite often the adhesion formation requires another operation just to eliminate the adhesions, though there is no guarantee against the adhesion formation after re-operation. One means of eliminating adhesion is to separate the tissues affected during surgery with some material which prevents diffusion of fibrinogen into the space between the tissues thus eliminating the formation of continuous fibrin clots in the space. A biocompatible viscoelastic gel slurry can be successfully used as an adhesion preventing material. However, the diffusion of low and high molecular weight substances in the case of plain gel slurries can easily occur between gel particles especially when the slurry mixes with body fluids and gel particles are separated from each other. On the other hand, when a viscoelastic mixed gel slurry according to one embodiment, is implanted into the body, the polymer solution phase located between gel particles continues to restrict the diffusion even after dilution with body fluids thus preventing adhesion. Moreover, this effect would be more pronounced with an increase in polymer concentration of the polymer solution phase.

The same is true when the viscoelastic mixed gel slurries according to one embodiment are used as drug delivery vehicles. Each of the phases of the slurry or both phases can be loaded with a drug or any other substance having physiological activity which will slowly diffuse from the viscoelastic slurry after its implantation into the body and the diffusion rate can be conveniently controlled by changing the compositional parameters of the slurries.

Components of the viscoelastic mixed gel slurries according to one embodiment affect the behavior of living cells by slowing down their movement through the media and preventing their adhesion to various surfaces. The degree of manifestation of these effects depends strongly on such factors as the composition of the two components of the mixture and their ratio, the nature of the surface and its interaction with the viscoelastic gel slurry, type of the cells, etc. But in any case this property of the viscoelastic gel slurries can be used for treatment of medical disorders where regulation of cell movement and attachment are of prime importance in cases such as cancer proliferation and metastasis.

In addition to the above two applications of biocompatible viscoelastic gel slurries according to one embodiment other possible applications include soft tissue augmentation, use of the material as a viscosurgical tool in opthalmology, otolaryngology and other fields, wound management, in orthopedics for the treatment of osteoarthritis, etc. In all of these applications the following basic properties of the mixed gel slurries are utilized: biocompatibility, controlled viscoelasticity and diffusion characteristics, easily controlled residence time at the site of implantation, and easy handling of the material allowing, for example its injection through a small diameter needle. The following methods were used for characterization of the products obtained according to one embodiment. The concentration of hylan or hyaluronan in solution was determined by hexuronic acid assay using the automated carbazole method (E. A. Balazs, et al, Analyt. Biochem. 12, 547-558, 1965). The concentration of hylan or hyaluronan in the gel phase was determined by a modified hexuronic acid assay as described in Example 1 of U.S. Pat. No. 4,582,865.

Rheological properties were evaluated with the Bohlin Rheometer System which is a computerized rheometer with controlled shear rate and which can operate in three modes: viscometry, oscillation and relaxation. The measurements of shear viscosity at low and high shear rates characterize viscous properties of the viscoelastic gel slurries and their pseudoplasticity (the ratio of viscosities at different shear rates) which is important for many applications of the products. Measurements of viscoelastic properties at various frequencies characterized the balance between elastic (storage modulus G′) and viscous (loss modulus G″) properties. The relaxation characteristics were evaluated as the change of the shear modulus G with time and expressed as the ratio of two modulus values at different relaxation times.

Next, various HA Crosslinking Approaches are discussed. The following reactions focus mainly on the two most reactive functional groups—the hydroxyl and the carboxyl.
1.1. Bisepoxide,
   Ethyleneglycol diglycidyl ether
   1,4-butanediol diglycidyl ether
   This method was originally developed to crosslink agarose. Currently to crosslink HA the reaction is in dilute NaOH using bisepoxybutane and sodium borohydride. Reaction of hyaluronan with ethyleneglycol diglycidyl ether in ethanolic 0.1 N NaOH at 60° C. also afforded a hydrogel (FIG. 4A). The resulting gels had high water contents (>95%) and were investigated for use as an inflammation (stimulus)-responsive degradable matrix for implantable drug delivery. A hydrogel prepared from hyaluronan and alkaline 1,4-butanediol diglycidyl ether was highly porous. This material was then activated with perioxidate and then modified with an 18-amino acid peptide containing a cell attachment domain, Arg-Gly-Asp (RGD), to enhance cell attachment to the hydrogel. In alkaline medium, divinyl sulfone also cross-links hyaluronan, most likely via reaction with hydroxyl groups.

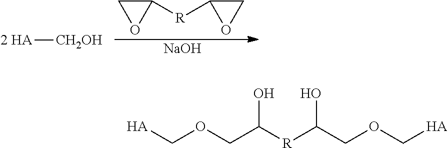

1.2. Divinylsulfone (DVS)
   In alkaline medium, divinyl sulfone also cross-links hyaluronan, most likely via reaction with hydroxyl groups.

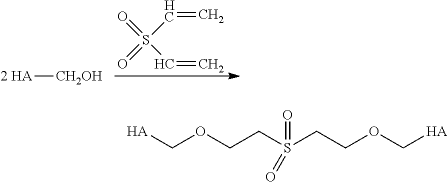

1.3. Internal esterification
   The autocross-linked polymer (ACP™, Fidia) is an internally esterified derivative of hyaluronan, with both inter- and intra-molecular bonds between the hydroxyl and carboxyl groups of hyaluronan. ACP™ can be lyophilized to a white powder and hydrated to a transparent gel. This novel biomaterial has been used as a barrier to reduce post-operative
1.4. Photo-cross Linking
   A methacrylate derivative of hyaluronan was synthesized by the esterification of the hydroxyls with excess methacrylic anhydride, as described above for hyaluronan butyrate. This derivative was photocross-linked to form a stable hydrogel using ethyl eosin in 1-vinyl-2-pyrrolidone and triethanolamine as an initiator under argon ion laser irradiation at 514 nm. The use of in situ photopolymerization of an hyaluronan derivative, which results in the formation of a cohesive gel enveloping the injured tissue, may provide isolation from surrounding organs and thus prevent the formation of adhesions. A preliminary cell encapsulation study was successfully performed with islets of Langerhans to develop a bioartificial source of insulin.
1.5. Glutaraldehyde cross linking
   Hyaluronan strands extruded from cation-exchanged sodium hyaluronate (1.6 MDa) were cross-linked in glutaraldehyde aqueous solution, although the chemical nature of this process was not identified. The strand surfaces were then remodeled by attachment of poly-D- and poly-L-lysine. The polypeptide-resurfaced hyaluronan strands showed good biocompatibility and promoted cellular adhesion.
1.6. Metal cation mediated cross linking
   Intergel® (FeHA, LifeCore) is a hydrogel formulation of hyaluronan formed by chelation with ferric hydroxide. Similar cross-linking of yaluronan has been the basis of preparations using copper, zinc, calcium, barium, and other chelating metals. The reddish FeHA gel is in development for prevention of post-surgical adhesions.
1.7. Carbodiimide cross linking
   Incert® is a bioresorbable sponge (Anika Therapeutics) prepared by cross-linking hyaluronan with a biscarbodiimide in aqueous isopropanol. This procedure takes advantage of the otherwise undesirable propensity of carbodiimides to react with hyaluronan to form N-acylureas. In this application, the formation of two N-acylurea linkages provides a chemically stable and by-product-free cross-link. Because of the hydrophobic biscarbodiimides employed, Incert® adheres to tissues without the need for sutures and retains its efficacy even in the presence of blood. Recently, it was found to be effective at preventing post-operative adhesions in a rabbit fecal abrasion study.

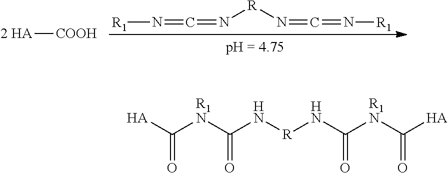

A low-water content hyaluronan hydrogel film was made by cross-linking a hyaluronan (1.6 MDa) film with a water-soluble carbodiimide as a coupling agent in an aqueous mixture containing a water-miscible non-solvent of hyaluronan. The highest degree of cross-linking that gave a low-water content hydrogel was achieved in 80% ethanol. This film, having 60% water content, remained stable for two weeks after immersion in buffered solution. The cross-linking of hyaluronan films with a water-soluble carbodiimide in the presence of L-lysine methyl ester further prolonged the in vivo degradation of a hyaluronan film.
1.8. Hydrazide cross linking
   Using the hydrazide chemistry described above, hydrogels have been prepared using bishydrazide, trishydrazide, and polyvalent hydrazide compounds as cross-linkers. By adjusting the reaction conditions and the molar ratios of the reagents, gels with physicochemical properties ranging from soft-pourable gels to more mechanically-rigid and brittle gels could be obtained. HA-ADH can be cross-linked using commercially-available small molecule homobifunctional cross-linkers
   More recently, an in situ polymerization technique was developed by cross-linking HA-ADH with a macromolecular cross-linker, PEG-dialdehyde under physiological conditions.
   Biocompatible and biodegradable hyaluronan hydrogel films with well-defined mechanical strength were obtained after the evaporation of solvent. Macromolecular drugs were released slowly from these hyaluronan hydrogel films, and these new materials accelerated re-epithelialization during wound healing.

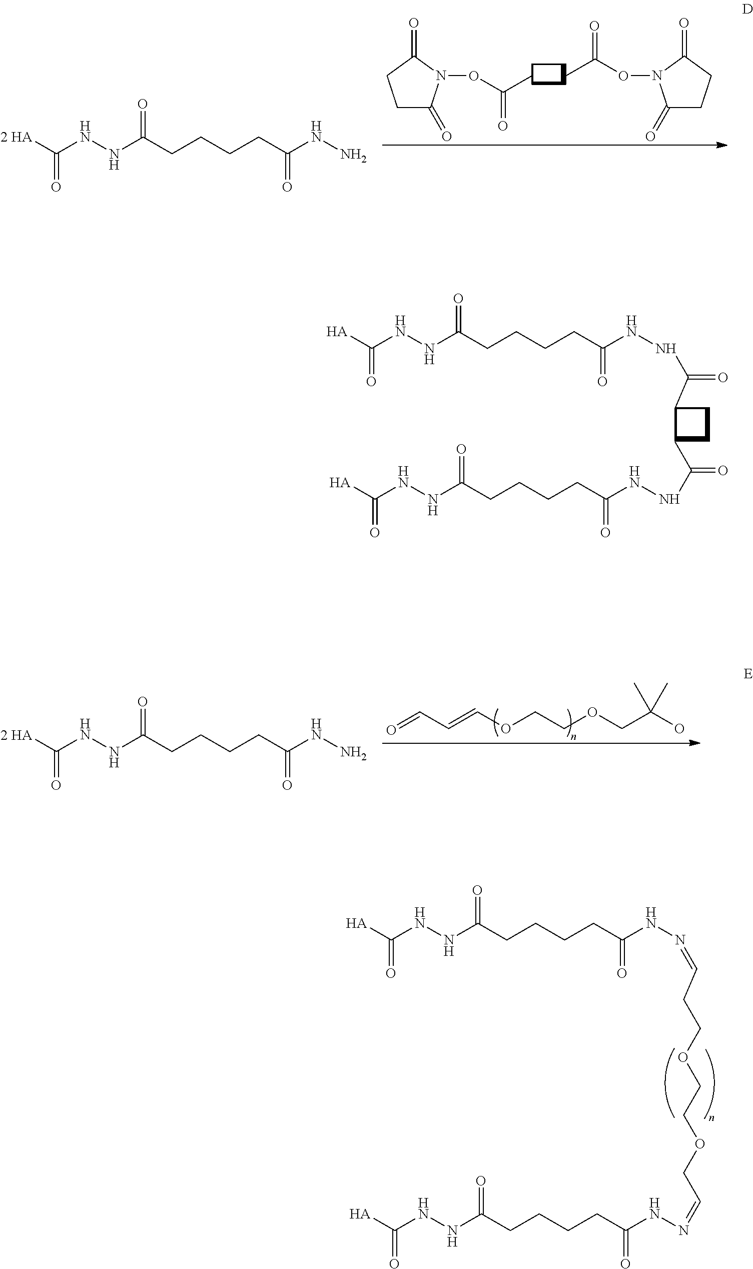

1.1. Cross linking with residual proteins
   Example of this is Hylans (Biomatrix) are hydrogels or hydrosols formed by cross-linking hyaluronan-containing residual protein with formaldehyde in a basic solution. 13 Soluble hylan is a high molecular weight form (8-23 MDa) of hyaluronan that exhibits enhanced rheological properties compared to hyaluronan. Hylan gels have greater elasticity and viscosity than soluble hylan materials, while still retaining the high biocompatibility of native hyaluronan. Hylans have been investigated in a number of medical applications.
1.2. Multi-component reactions
   These are 3 to 4 component reactions known as (1) the Passerini reaction and (2) Ugi reactions.
   In the Passerini reaction, an aqueous solution of hyaluronan is mixed with aqueous glutaraldehyde (or another water-soluble dialdehyde) and added to a known amount of a highly reactive isocyanide, e.g., cyclohexylisocyanide.
   In the Ugi four-component reaction (FIG. 4F), a diamine is added to this three-component mixture.
   The degree of cross-linking is controlled by the amount of aldehyde and diamine.

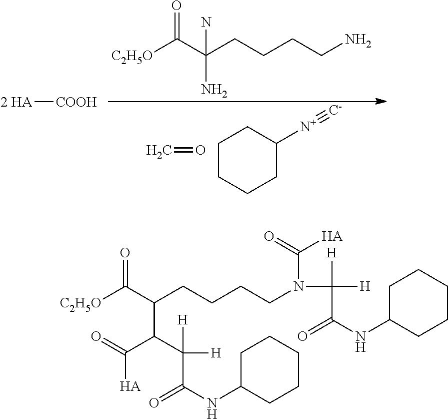

1.3. Surface modifications
   One example has to do with the Surfaces of polypropylene (PP) and polystyrene (PS) were activated with argon gas and ammonia gas plasmas to emanate the polymer surface. Emanated surfaces were then modified with succinic anhydride to give pendant carboxylic acid groups on the surface, which were then condensed with HA-ADH in the presence of a carbodiimide to give hydrophilic, non-adhesive, and lubricious plastic surfaces. Metal and glass surfaces can also be modified by surface activation followed by covalent chemical attachment of an appropriate hyaluronan derivative.

2. There are four different therapeutic modification options for HA as shown below
2.1. A: HA can be cross-linked at two locations: (1) the hydroxyl location and (2) the carboxyl location.
2.2. B: Drugs that have functional groups that favor reacting with hydroxyl and/or carboxyl could be conjugated on the HA molecule, and the HA molecule will act as a carrier of the drug.
2.3. C: Individual HA molecules could be grafted or attached covalently to a polymer chain that has pendant function groups which favor reacting with hydroxyl and/or carboxyl.
2.4. D. HA molecules can be grafted onto a liposome provided that their function groups favor reacting.

HA Therapeutic Modification Options
Include cross-linked HA hydrogel, HA drug bioconjugate, HA-grafted copolymers, and HA liposomes

HA Reactive Sites

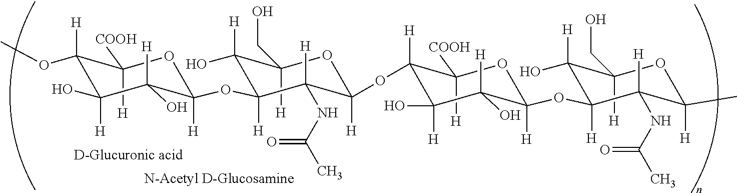

2.5. Carboxyl group chemical reactions
   2.5.1. Esterification

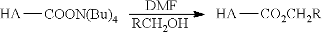

Esterified hyaluronan biomaterials have been prepared by alkylation of the tetra (n-butyl) ammonium salt of hyaluronan with an alkyl halide in dimethylformamide (DMF) solution. These hyaluronan esters can be extruded to produce membranes and fibers, lyophilized to obtain sponges, or processed by spray-drying, extraction, and evaporation to produce microspheres. These polymers show good mechanical strength when dry, but the hydrated materials are less robust. The degree of esterification influences the size of hydrophobic patches, which produces a polymer chain network that is more rigid and stable, and less susceptible to enzymatic degradation.
2.5.2. Carbodiimide-mediated reactions

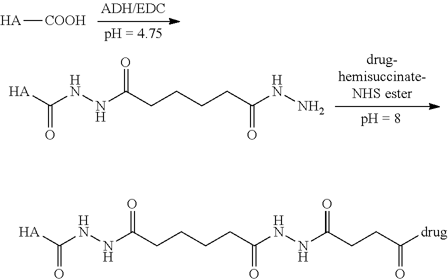

2.5.3. The chemical modification of the carboxylic functions of hyaluronan by carbodiimide compounds is generally performed in water at pH 4.75.
2.6. Hydroxyl group chemical reactions
   2.6.1. Sulfation
      The sulfation of hyaluronan with a sulfur trioxide-pyridine complex in DMF produced different degrees of sulfation, HyalSx, where x=1-4 per disaccharide. The sulfated hyaluronic acid HyalS3.5 was then immobilized onto plasma-processed polyethylene (PE) using a diamine polyethylene glycol derivative and a water-soluble carbodiimide. The thrombin time test and platelet adhesion behavior indicated that this procedure was promising for the preparation of blood-compatible, anti-thrombotic PE surfaces. In addition, HyalSx was converted to a photo labile azidophenylamino derivative and was photoimmobilized onto a poly(ethylene terephthalate) (PET) film.9 Surfaces coated with sulfated hyaluronan exhibited marked reduction of cellular attachment, fouling, and bacterial growth compared with uncoated surfaces, and the coating was stable to degradation by chondroitinase and hyaluronidase.
      Hyaluronan butyrate is used as targeted drug-delivery system specifically to tumor cells. Butyric acid is known to induce cell differentiation and to inhibit the growth of a variety of human tumors was coupled to hyaluronan via the reaction between butyric anhydride and the sym-collidinium salt of low molecular weight hyaluronan in DMF containing dimethylaminopyridine.
   2.6.2. Isourea coupling or cyanogen bromide activation
      The anthracycline antibiotics adriamycin and daunomycin were coupled to hyaluronan via cyanogen bromide (CNBr) activation. This reaction scheme is commonly used to activate oligosaccharides to produce affinity matrices via a highly-reactive isourea intermediate. The therapeutic agents appear to become attached via a urethane bond to one of the hydroxylic functions of the oligosaccharide or the glycosaminoglycan, but no spectroscopic verification was provided. Moreover, the harshness of the reaction conditions may compromise the integrity and biocompatibility of the hyaluronan.

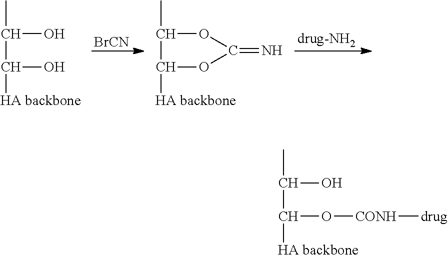

2.6.3. Peroxidase oxidation
   Reactive bisaldehyde functionalities can be generated from the vicinal secondary alcohol functions on hyaluronan by oxidation with sodium peroxidase. This chemistry is a standard method for chemical activation of glycoproteins for affinity immobilization or conversion to a fluorescent probe. With peroxidase-activated hyaluronan, reductive coupling with primary amines can give cross-linking, attachment of peptides containing cell attachment domains, or immobilized materials. The harsh oxidative treatment also introduces chain breaks and potentially immunogenic linkages into the hyaluronan biomaterial.

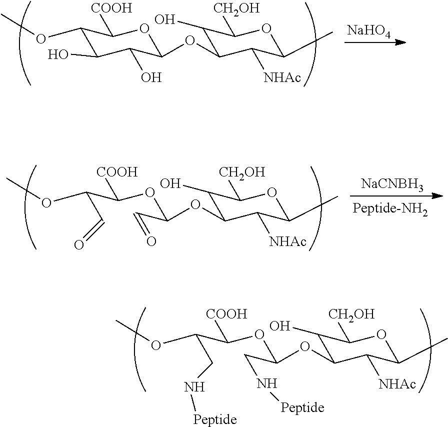

2.6.4. Reducing end modification
   Reductive amination of the reducing end of hyaluronan has been employed to prepare affinity matrices, fluorophore-labeled materials, and hyaluronan-phospholipids for insertion into hyaluronan-liposomes. For example, low molecular weight hyaluronan was covalently attached to phosphatidyl-ethanolamine, and this conjugate has been employed for a protective “sugar decoration” on the surface of low density lipoprotein (LDL) particles. End-labeling has not otherwise been extensively used for hyaluronan biomaterials or pro-drug applications, since there is only one attachment point per glycosaminoglycan. This severely limits loading and cross-linking possibilities for high molecular weight hyaluronan.
2.6.5. Amide modifications
   Native hyaluronan has, in some preparations, an undetermined number of naturally deacylated glucosamine units that may also be derivatized. As with the reducing end modification, this provides very low modification rates. However, modification of the N-acetyl groups can be important if the commonly used hydrazinolysis method is employed. Limited hydrazinolysis of hyaluronan creates free glucosamine residues on hyaluronan, but can also result in base-induced backbone cleavage and reducing end modification.
      In yet other experiments, the Materials can include

1.1. Hyaluronic Acid, sodium salt, streptococcus equi, Phosphate buffered saline

1.2. 1,4-butanediol diglycidyl ether (BDDE)

1.3. Divinyl sulfone

1.4. Sodium hydroxide pellets

1.5. De-ionized water

1.6. Analytical scale

1.7. Microliter pipette

1.8. Microliter syringe

1.9. Standard lab equipment

### 2. Experimental Methods

2.1. Experiment 001-12: Water in oil emulsion cross-linking reaction

Aqueous phase mix

| COMPONENTS | Quantity |
|---|---|
| Hyaluronic acid sodium | 6.5% |
| NaOH | 2M |
| Make total final volume | 0.54 mL |

Oil phase mix

| COMPONENTS | AMOUNT | |
|---|---|---|
| Isooctane | 13 | mL |
| Sodium-bis-sulfosucinate 0.2M | 1 | mL |
| Trimethylpentane 0.04M | 1 | mL |
| DVS | 45 | μL |

2.1.1. The reaction is a water in oil emulsion reaction
2.1.2. Let it react at RT for 1 hour
2.1.3. Collect the gel particles by centrifuge
2.1.4. Wash with acetone

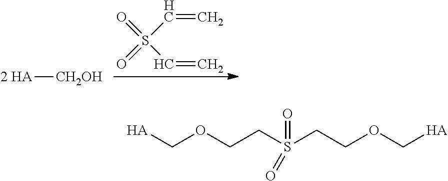

2.2. Experiment 001-14

Reaction Mixture

| COMPONENTS | AMOUNT |
|---|---|
| Hyaluronic acid | 0.105 g |
| X-Linker Mix: a, b, c, d and e | 0.775 g |

X-Linker Mix

| | AMOUNT | | | | |
|---|---|---|---|---|---|
| COMPONENTS | a | b | c | d | e |
| NaOH 1% | 9.99 | 9.98 | 9.97 | 9.96 | 9.95 |
| BDDE | .010 | .020 | .030 | .040 | .050 |

2.2.1. The X-Linker mix is made up first
2.2.2. Make up the reaction mixture next
2.2.3. Add 0.775 g of the x-linker mix “a” through “e” to the HA. There are reactions.
2.2.4. Mix well with a spatula to work the x-linker into the HA
2.2.5. Let each reaction take place at RT with mixing every 30-60 min
2.2.6. After 8 hours of reacting the product is a cross linked hyaluronic acid gel
2.2.7. Placed into a 52 C for 3 hours with mixing every 0.5 hours
2.2.8. Washed 3× with PBS

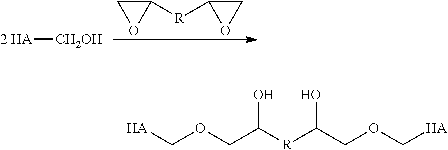

2.3. Boundary Conditions of Components in the HA X-Linking Process
2.3.1. Experiment 001-16: X-Linker mix storage life and Reaction Temperature
   2.3.1.1. The X-linker mix must be used sooner than 24 hours after made up and kept at RT conditions
   2.3.1.2. The reaction temperature of 50 C is too high to be kept for more than 1 hour.
2.3.2. Experiment 001-17: Storage life for 1% NaOH
   2.3.2.1. NaOH solution containing x-linker should be used with 1 hour of its preparation
   2.3.2.2. NaOH concentration of 1 normal is too low to yield completely reacted product
2.3.3. X-Linker Storage Life—BDDE
   2.3.3.1. Experiment 001-18: Showed that once mixed with NaOH, the mixture containing BDDE should be used within 3 hours.

2.4. X-Linker Storage Life—DVS “TBD”

2.5. Experiment 001-19

| | |
|---|---|
| COMPONENTS | AMOUNT |

Mixture A

| | | |
|---|---|---|
| Empty culture tube | 8.755 | g |
| HA | 0.105 | g |
| NaOH 1N | 0.5 | mL |

Mixture B

| | | |
|---|---|---|
| NaOH 1N | 2 | mL |
| BDDE | 0.02 | mL |

Final Mixture

| COMPONENTS | AMOUNT |
|---|---|
| Mixture A | All |
| Mixture B | 0.5 mL |

2.5.1. Mix well after added A and B together
2.5.2. Let Stand at RT for 2 hours with mixing every 30 min
2.5.3. Let stand in 50 C for 1 hour with mixing every 30 min
2.5.4. Product looks very much like commercial product, Juvéderm

2.6. Experiment 001-21

| | |
|---|---|
| COMPONENTS | AMOUNT |

Mixture A

| | | |
|---|---|---|
| Empty culture tube | 10.510 | g |
| HA | 0.105 | g |
| NaOH 1% | 0.5 | mL |

Mixture B

| | |
|---|---|
| NaOH 1% | 9.9 |
| DVS (divinyl sulfone) | .010 |

Final Mixture

| COMPONENTS | AMOUNT |
|---|---|
| Mixture A | All |
| Mixture B1-B5 | 0.775 mL |

2.6.1. Mix well after added A and B1 through B5 respectively together
2.6.2. Let Stand at RT for 2 hours with mixing every 30 min
2.6.3. Let stand in 50 C for 1 hour with mixing every 30 min
2.6.4. Product looks very much like a commercial product, Juvéderm

2.7. Effects of X-Linking Levels
2.7.1. Experiment 001-22: BDDE (1,4-butanediol diglycidylether)

| | |
|---|---|
| COMPONENTS | AMOUNT |

(HA Mix) × 4

| | | |
|---|---|---|
| HA | 0.105 | g |
| X-Linker Mix _{——} | 0.775 | mL |

BDDE Mix A

| | | |
|---|---|---|
| NaOH 1% | 9.99 | mL |
| BDDE | 0.01 | mL |

BDDE Mix B

| | | |
|---|---|---|
| BDDE Mix A | 1 | mL |
| NaOH 1% | 1 | mL |

BDDE Mix C

| | | |
|---|---|---|
| BDDE Mix A | 1 | mL |
| NaOH 1% | 2 | mL |

BDDE Mix D

| | | |
|---|---|---|
| BDDE Mix A | 1 | mL |
| NaOH 1% | 3 | mL |

2.7.2. Experiment 001-25: DVS (Divinyl sulfone)

| | |
|---|---|
| COMPONENTS | AMOUNT |

(HA Mix) × 4

| | | |
|---|---|---|
| HA | 0.130 | g |
| X-Linker Mix _{——} | 0.800 | mL |

DVS Mix A

| | | |
|---|---|---|
| NaOH 1% | 9.99 | mL |
| DVS (divinyl sulfone) | 0.01 | mL |

DVS Mix B

| | | |
|---|---|---|
| DVS Mix A | 1 | mL |
| NaOH 1% | 1 | mL |

DVS Mix C

| | | |
|---|---|---|
| DVS Mix A | 1 | mL |
| NaOH 1% | 2 | mL |

DVS Mix D

| | | |
|---|---|---|
| DVS Mix A | 1 | mL |
| NaOH 1% | 3 | mL |

The other methods, used for characterization of the products according to one embodiment are described in the following examples which illustrate preferred embodiments of one embodiment without, however, being a limitation thereof.

In one implementation, a method for producing an HA gel slurry includes forming an inner core using a non-biological synthesis process; and encapsulating the inner core with an hyaluronic acid (HA) or glycosaminoglycan (GAG) made from a biological synthesis process. The inner core can be genetic or metabolic engineering for HA synthesis. The inner core can also be artificial (in vitro) synthesis of HA by enzymes. The inner core can also be cellulose, polysaccharide, hydroxypropyl cellulose, among others. The use of non-biologically synthesized core and a biologically produced HA allows large volumes of HA to be made in an economical manner while maintaining biological compatibility. Thus, safer, purer and more consistent hyaluronic acid material can be produced quickly and economically. Such system would coat the expensive animal tissue extracts in the tissue interface or contacting area, while less expensive materials can be used in the core of the entire material to provide longevity.

An exemplary method for producing an HA gel slurry includes 1) forming an inner core using a non-biological synthesis process; and 2) forming an hyaluronic acid (HA) or glycosaminoglycan (GAG) using a biological synthesis process. The use of non-biologically synthesized core and a biologically produced HA allows large volumes of HA to be made in an economical manner while maintaining biological compatibility. Thus, safer, purer and more consistent hyaluronic acid material can be produced quickly and economically. Such system would coat the expensive animal tissue extracts in the tissue interface or contacting area, while less expensive materials can be used in the core of the entire material to provide longevity.

The hyaluronan of a recombinant Bacillus cell is expressed directly to the culture medium in one embodiment. In this embodiment, a simple process may be used to isolate the hyaluronan from the culture medium. First, the Bacillus cells and cellular debris are physically removed from the culture medium. The culture medium may be diluted first, if desired, to reduce the viscosity of the medium. Many methods are known to those skilled in the art for removing cells from culture medium, such as centrifugation or microfiltration. If desired, the remaining supernatant may then be filtered, such as by ultrafiltration, to concentrate and remove small molecule contaminants from the hyaluronan. Following removal of the cells and cellular debris, a simple precipitation of the hyaluronan from the medium is performed by known mechanisms. Salt, alcohol, or combinations of salt and alcohol may be used to precipitate the hyaluronan from the filtrate. Once reduced to a precipitate, the hyaluronan can be easily isolated from the solution by physical means. The hyaluronan may be dried or concentrated from the filtrate solution by using evaporative techniques known to the art, such as lyophilization or spray drying.

In one embodiment, the inner core can be bio-compatible composition such as polymers: silicones, poly (ethylene), poly (vinyl chloride), polyurethanes, polylactides. The inner core can also be natural polymers: collagen, gelatin, elastin, silk, polysaccharide. The inner core can also be cellulose, polysaccharide, hydroxypropyl cellulose, among others. The inner core can be genetic or metabolic engineering for HA synthesis. The inner core can also be artificial (in vitro) synthesis of HA by enzymes. One implementation for making the inner core of synthetic hyaluronic acid employs two monosaccharide glycosyl donors to create the repeating polymer. The reverse disaccharide pathway provides a hyaluronic acid-like glucose-β-(1→4)-glucosamine disaccharide.

Cellulose is an organic compound with the formula (C6H10O5)n, a polysaccharide consisting of a linear chain of several hundred to over ten thousand β(1→4) linked D-glucose units. Cellulose is an important structural component of the primary cell wall of green plants, many forms of algae and the oomycetes. Some species of bacteria secrete it to form biofilms.[4] Cellulose is the most abundant organic polymer on Earth.[5] The cellulose content of cotton fiber is 90%, that of wood is 40-50% and that of dried hemp is approximately 45%. Cellulose is mainly used to produce paperboard and paper. Smaller quantities are converted into a wide variety of derivative products such as cellophane and rayon. Conversion of cellulose from energy crops into biofuels such as cellulosic ethanol is under investigation as an alternative fuel source. Cellulose for industrial use is mainly obtained from wood pulp and cotton.

Carbohydrates (saccharides) are divided into four chemical groups: monosaccharides, disaccharides, oligosaccharides, and polysaccharides. In general, the monosaccharides and disaccharides, which are smaller (lower molecular weight) carbohydrates, are commonly referred to as sugars.[6] The word saccharide comes from the Greek word σ\acute{α}κχαρo ν (sákkharon), meaning “sugar.” While the scientific nomenclature of carbohydrates is complex, the names of the monosaccharides and disaccharides very often end in the suffix-ose. For example, grape sugar is the monosaccharide glucose, cane sugar is the disaccharide sucrose, and milk sugar is the disaccharide lactose.

Carbohydrates perform numerous roles in living organisms. Polysaccharides serve for the storage of energy (e.g., starch and glycogen), and as structural components (e.g., cellulose in plants and chitin in arthropods). The 5-carbon monosaccharide ribose is an important component of coenzymes (e.g., ATP, FAD, and NAD) and the backbone of the genetic molecule known as RNA. The related deoxyribose is a component of DNA. Saccharides and their derivatives include many other important biomolecules that play key roles in the immune system, fertilization, preventing pathogenesis, blood clotting, and development.

Hydroxypropyl cellulose (HPC) is a derivative of cellulose with both water solubility and organic solubility. It is used as a topical ophthalmic protectant and lubricant. HPC is an ether of cellulose in which some of the hydroxyl groups in the repeating glucose units have been hydroxypropylated forming —OCH2CH(OH)CH3 groups using propylene oxide. The average number of substituted hydroxyl groups per glucose unit is referred to as the degree of substitution (DS). Complete substitution would provide a DS of 3. Because the hydroxypropyl group added contains a hydroxyl group, this can also be etherified during preparation of HPC. When this occurs, the number of moles of hydroxypropyl groups per glucose ring, moles of substitution (MS), can be higher than 3. Because cellulose is very crystalline, HPC must have an MS about 4 in order to reach a good solubility in water. HPC has a combination of hydrophobic and hydrophilic groups, so it has a lower critical solution temperature (LCST) at 45° C. At temperatures below the LCST, HPC is readily soluble in water; above the LCST, HPC is not soluble. HPC forms liquid crystals and many mesophases according to its concentration in water. Such mesophases include isotropic, anisotropic, nematic and cholesteric.

Synthesis of hyaluronan using isolated HA synthase can be done when hyaluronan polymers of defined molecular weight and narrow polydispersity are needed. IsolatedHAsynthase is able to catalyze in vitro at well-defined conditions the same reaction as it catalyzes in vivo, namely, the synthesis of hyaluronan from the nucleotide sugars UDPGlcNAc andUDP-GlcUA. Preparative enzymatic synthesis of hyaluronan using the crude membrane-bound HA synthase from S. pyogenes was demonstrated, although the yield was low, around 20%[105]. Thehyaluronan yield was increased to 90% when the enzymatic hyaluronan synthesis was coupled with in situ enzymatic regeneration of the sugar nucleotides using UDP and relatively inexpensive substrates, Glc-1-P and GlcNAc-1-P in a one-pot reaction. The average molecular weight of the synthetic hyaluronan was around 5.5×105 Da, corresponding to a degree of polymerization of 1500. High molecular weight monodisperse hyaluronan polymers with Mw up to 2.500 kDa (12,000 sugar units) and polydispersity (Mw/Mn) of 1.01-1.20 were obtained by enzymatic polymerization using the recombinant P.multocidaHA synthase, PmHAS, overexpressed in E. coli. PmHAS uses two separate glycosyl transferase sites to add GlcNAc and GlcUAmonosaccharides to the nascent polysaccharide chain. Hyaluronan synthesis with PmHAS was achieved either by de novo synthesis from the two UDP-sugars precursors (1) and by elongation of an hyaluronan-like acceptor oligosaccharide chain by alternating, repetitive addition of the UDP-sugars as follows: nUDP-GlcUA+nUDP-GlcNAc+z[GlcUA-GlcNAc]x □→2nUDP+[GlcUA+GlcNAc]x+n. The control of the chain length and polydispersity of the hyaluronan polymer is determined by the intrinsic enzymological properties of the recombinant PmHAS, (i) the rate limiting step of the in vitro polymerization appears to be the chain initiation, and (ii) in vitro enzymatic polymerization is a fast nonprocessive reaction. Therefore, the concentration of the hyaluronan acceptor controls the size and the polydispersity of the hyaluronan polymer in the presence of a finite amount of UDP-sugar monomers [106]. Using this synchronized, stoichiometrically-controlled enzymatic polymerization reaction, low molecular weight hyaluronan (˜8 kDa) with narrow size distribution was synthesized. One important feature of the PmHAS is that chain elongation occurs at the nonreducing end of the growing chain and this makes the use of modified acceptors as substrates possible and consequently the synthesis of hyaluronan polymers with various end-moieties. The elongation of a hyaluronan tetrasaccharide labeled at the reducing end with the fluorophore 2-aminobenzoic acid, among others.

Variations and modifications can, of course, be made without departing from the spirit and scope of the invention.

What is claimed is:

1. A method for producing a gel slurry having a plurality of cross-linked units each formed by:
providing an inner core using a non-biological synthesis process; and
cross-linking at a first cross-link strength using a hyaluronic acid (HA) or glycosaminoglycan (GAG) made from a biological synthesis process followed by additional cross-linkings at a second cross-link strength with HA or GAG, wherein the first cross-link strength is stronger than the second cross-link strength.

2. The method of claim 1, wherein the inner core comprises genetic or metabolic engineering for HA synthesis.

3. The method of claim 1, wherein the inner core comprises artificial (in vitro) synthesis of HA by enzymes.

4. The method of claim 1, wherein the inner core comprises cellulose, polysaccharide, hydroxypropyl cellulose, collagens, protein, an extracellular matrix of a biological system.

5. The method of claim 1, comprising:
cross-linking the HA to form a single cross-linked material; and
performing one or more additional cross-linkings on the single cross-linked material to form a multiple cross-linked material,
wherein the multiple cross-linked material has one or more IPN regions resisting biodegradation in a human body than the single cross-linked material and one or more single cross-linked extensions radiating out from the IPN, wherein the combination of the IPN and the extension provide one or more of: biodegradation resistance, soft touch feeling, ease of insertion into the human body.

6. The method of claim 1, comprising encapsulating the inner core using DVS for tight cross-linking with a hyaluronic acid (HA) or glycosaminoglycan (GAG) made from a biological synthesis process, further comprising weak cross-linking with HA or GAG using BDDE.

7. The method of claim 1, comprising using a syringe or a mechanical pump to inject the biocompatible cross-linked polymer in a breast or a buttock or under soft tissue in a minimally invasive manner.

8. The method of claim 1, comprising applying the gel slurry to an exterior of a breast implant with the HA or GAG facing patient tissues to improve biocompatibility.

9. The method of claim 1, comprising using cross linkers and forming thermoset polymers or to form cross linked copolymers by crosslinking with other polymer species using multifunctional monomers.

10. The method of claim 1, comprising forming with a biocompatible viscoelastic gel slurry comprising a two phase mixture, a first phase being a non-biologically produced gel phase and a second phase being a biologically produced, biocompatible gel phase, said biocompatible gel phase comprising a chemically cross-linked glycosaminoglycan that is multiply-cross-linked.

11. The method of claim 1, comprising adding a substance to the composition for biocompatibility.

12. The method of claim 1, comprising controlling drug releases at predetermined timing according physiological events.

13. The method of claim 12, comprising carrying the drug by biocompatible and biodegradable polymers.

14. The method of claim 1, comprising dispensing the drug uniformly throughout a material matrix of the biodegradable polymer.

15. The method of claim 1, wherein the inner core comprises an alcohol based synthetic polymer.

16. The method of claim 1, comprising providing a polymer that carries the drug including one of: polylactide (PLA), polyglycolide (PGA) and copolymers of PLA/PGA tailored to meet mechanical performance and resorption rates required for applications ranging from non-structural drug delivery polymer applications to biodegradable screws or anchors.

17. The method of claim 1, comprising releasing drug into a biological environment at the same rate as a polymer rate of degradation and the rate of drug diffusing from a polymer matrix.

18. The method of claim 1, comprising blending a drug carrier polymer composition and a filler polymer composition at a predetermined ratio.

19. The method of claim 1, comprising adding one or more of: an anesthetics, a lidocaine, a compound to reduce or eliminate acute inflammatory reactions, or a composition selected from the group consisting of steroids, corticosteroids, dexamethasone, triamcinolone.

20. The method of claim 1, wherein the gel slurry is injectable with a syringe for at least 100 cc of the biocompatible cross-linked polymer into a breast or a buttock in a minimally invasive manner.
